(19) United States
(12) Patent Application Publication
(10) Pub. No.: US 2011/0159087 A1
(43) Pub. Date: Jun. 30, 2011

(54) Crosslinked Polymers

(76) Inventors: Dhananjay Govind Sathe, Mumbai (IN); Harish Kashinath Mondkar, Mumbai (IN); Mantripragada Narayan Rao, Mumbai (IN); Samadhan Daulat Patil, Mumbai (IN); Tanaji Shamrao Jadhav, Mumbai (IN); Ashok Omray, Mumbai (IN); Varsha Shashank Choudhary, Mumbai (IN); Yogesh Sharad Bhide, Mumbai (IN)

(21) Appl. No.: 13/060,624

(22) PCT Filed: Sep. 2, 2009
(86) PCT No.: PCT/IN09/00483

§ 371 Date: Feb. 24, 2011

(30) Foreign Application Priority Data
Sep. 2, 2008 (IN) 1860/MUM/2008

(51) Int. Cl. A61K 9/48; A61K 31/785; A61K 9/14; A61K 9/20; C08G 73/02; A61P 3/00; B02C 23/18; B32B 5/00; A61K 9/28; B29C 43/02; B65B 1/20

(52) U.S. Cl. 424/452; 424/78.38; 424/78.16; 424/78.35; 424/400; 424/465; 525/404; 525/328.4; 241/15; 428/402; 427/2.14; 264/109; 141/12

(57) ABSTRACT

Disclosed herein are pharmaceutical compositions comprising wet granulated bile acid sequestrants having the general Formula I shown, and their process of preparation. The present invention also discloses process for preparation of Colesevelam hydrochloride, an antilipemic agent.

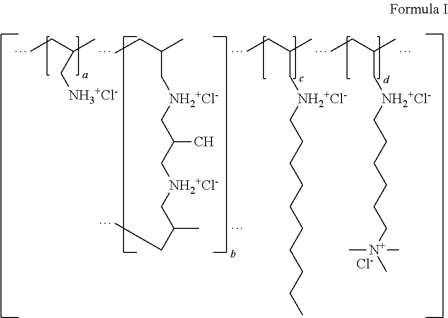

### RELATED APPLICATION

This application is a National Stage entry of PCT/1N2009/000483 filed on Sep. 2, 2009, which in turn claims priority from Indian Provisional Application No. 1860/MUM/2008 filed on Sep. 2, 2008.

### TECHNICAL FIELD

The present invention relates to pharmaceutical compositions comprising wet granulated bile acid sequestrants and their process of preparation. The bile acid sequestrant is a crosslinked polymer selected from the group consisting of Colesevelam, Cholestyramine, Colestipol, Sevelamer and Colestimide. The present invention further relates to the process for preparation of bile acid sequestrants, preferably Colesevelam hydrochloride, an antilipemic agent.

### BACKGROUND OF THE INVENTION

Colesevelam hydrochloride is a non-absorbed, polymeric, lipid-lowering and glucose-lowering agent intended for oral administration. Colesevelam hydrochloride is a high-capacity bile acid-binding molecule. The chemical name (IUPAC) of Colesevelam hydrochloride is allylamine polymer with 1-chloro-2,3-epoxypropane, [6-(allylamino)-hexyl]trimethylammonium chloride and N-allyldecylamine, hydrochloride of Formula I,

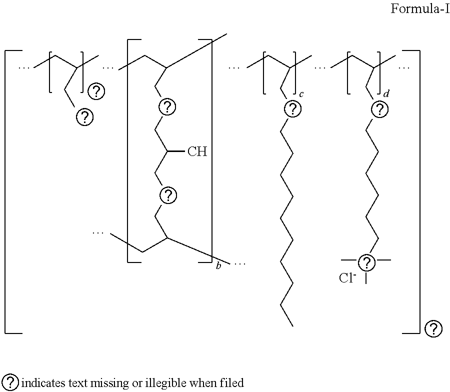

wherein (a) represents allyl amine monomer units that have not been alkylated by either of the 1-bromodecane or (6-bromohexyl)-trimethylammonium bromide alkylating agents or cross-linked by epichlorohydrin; (b) represents allyl amine units that have undergone crosslinking with epichlorohydrin; (c) represents allyl amine units that have been alkylated with a decyl group; (d) represents allyl amine units that have been alkylated with (6-trimethylammonium) hexyl group, and m represents a number ≧100 to indicate an extended polymer network. A small amount of the amines are dialkylated, and are not depicted in the formula above. No regular order of the groups is implied by the structure; cross-linking and alkylation are expected to occur randomly along the polymer chains. A large amount of the amines are protonated. The polymer is depicted in the hydrochloride form; a small amount of the halides are bromide.

Colesevelam is developed by Genzyme. Colesevelam is marketed in United states by Genzyme and Daiichi Sankyo under the brand name WELCHOL®. Colesevelam is marketed in Europe under the brand name Cholestagel. WELCHOL® is supplied as off-white, oval, film-coated, solid tablet containing 625 mg Colesevelam hydrochloride. Inactive ingredients in WELCHOL® are magnesium stearate, microcrystalline cellulose, silicon dioxide, hydroxypropyl methylcellulose and acetylated monoglyceride.

Colesevelam is a bile acid sequestrant and is administered orally. It has been shown to lower LDL cholesterol a mean of 19% and a dose of 3.8 g/d. Colesevelam is indicated as an adjunct to diet and exercise to reduce elevated low-density lipoprotein cholesterol (LDL-C) in patients with primary hyperlipidemia as monotherapy or in combination with an hydroxymethyl-glutaryl-coenzyme A (HMG CoA) reductase inhibitor. Lipid-altering agents should be used in addition to a diet restricted in saturated fat and cholesterol when response to diet and non-pharmacological interventions alone has been inadequate. Colesevelam is also indicated as an adjunct to diet and exercise to improve glycemic control in adults with type 2 diabetes mellitus.

Colesevelam hydrochloride is a non-absorbed, lipid-lowering polymer which binds the bile acids in the intestine and impedes their reabsorption. As the bile acid pool becomes depleted, the hepatic enzyme, cholesterol 7-α-hydroxylase, is upregulated, which increases the conversion of cholesterol to bile acids. This causes an increased demand for cholesterol in the liver cells, resulting in the dual effect of increasing transcription and activity of the cholesterol biosynthetic enzyme, HMG-CoA reductase, and increasing the number of hepatic low-density lipoprotein receptors. These compensatory effects result in increased clearance of LDL-C from the blood, resulting in decreased serum LDL-C levels (www.fda.gov/cder/foi/label/2008/021176s0171b1.pdf).

WO/2001/028527 discloses a tablet core which comprises at least about 95% by weight of an aliphatic amine polymer and method of producing comprising the step of (1) hydrating the aliphatic amine polymer to the desired moisture level; (2) blending the aliphatic amine polymer with the excipients in amounts such that the polymer comprises at least about 95% by weight of the resulting blend; and (3) compressing the blend to form the tablet core.

WO2005065291 discloses tablet, capsule and sachet that have an aliphatic amine polymer-containing core having an enteric coating that targets the release of aliphatic amine polymers to one or more specific intestinal regions.

Prior art discloses various formulations of Colesevelam Hydrochloride by methods involving direct compression. The prior art teaches away from wet granulation as the polymer being hygroscopic, swells on contact with water. However, the inventors of the present invention have surprisingly succeeded in formulating bile acid sequestrants by wet granulation method.

U.S. Pat. No. 5,693,675 describes process for preparation of polyallylamine hydrochloride which comprises addition of crosslinked poly(allylamine), (6-bromohexyl) trimethylammonium bromide and 1-bromodecane to a stirred solution of methanol and sodium hydroxide followed by heating the mixture with stirring. The obtained solid was dried in a vacuum oven at 50° C. The chloride content of polymer prepared according to this process does not match with that of WELCHOL® tablet. Comparative analysis of Colesevelam HCl prepared by this process (U.S. Pat. No. 5,693,675) and WELCHOL® tablet is as follows,

| Sr. | | WELCHOL ™ | U.S. Pat. No. | Present |
|---|---|---|---|---|
| No. | Parameters | tablet | 5,693,675 | invention |
| 01 | BBC (mM/gm) | 4.0 | 3.07 | 4.12 |
| 02 | Chloride (meq/gm) | 5.96 | 2.03 | 5.45 |
| 03 | Bromide (meq/gm) | 0.09 | 0.35 | Less than |
| | | | | about 0.1 |

The chloride content obtained by following this process is in the range of 2.0 to 2.5 meq/gm. Thus it is observed that the chloride content of product obtained by the prior art process is not as per the requirement and the process is also not consistent in providing the desired chloride content.

U.S. Pat. No. 5,607,669 describes process for alkylating the cross linked poly-(allylamine) using 6-bromohexyl trimethylammonium bromide and 1-bromodecane in presence of aqueous base or methanolic solution of base. In this patent alkylation is carried out on gelled crosslinked amine polymer which is prepared in situ and further alkylated with alkylating agents. After completion of alkylation, the polymer was treated with methanol (4 times), 2M sodium chloride solution in water (6 times) and deionised water (6 times). Hence total 16 times washing treatment has been given to the polymer.

U.S. Pat. No. 7,148,319 describes process for the alkylation of crosslinked polymers which comprises a) optionally deprotonating the gelled polymers obtained by polymerization and crosslinking in water, an organic solvent or in an organic solvent/water mixture by addition of a base, b) optionally washing the polymers one or more times with water, an organic solvent or an organic solvent/water mixture, then c) adding one or more alkylators at atmospheric or elevated pressure at a temperature of between 5 and 160° C. to the gel suspension which is stirred in water, an organic solvent or in an organic solvent/water mixture and after a mixing time of 1 to 60 minutes adding the base continually or in plural portions in such a way that the pH is between 8 and 13.5 and d) subsequently carrying out the reprotonation by means of concentrated acid to get the product. In this patent, alkylation is carried out on gelled polymer Sevelamer which was prepared in situ. After completion of alkylation, polymer was treated with methanol (4 times), 2M sodium chloride solution in water (7 times) and deionized water (6 times).

U.S. Pat. No. 7,105,631 relates to an alkylation process which comprises cutting the crude gel into defined shape and washing the crosslinked polymer (Sevelamer) gel with methanol. The obtained mass was then alkylated in methanol by adding one or/and more alkylators at 5-90° C. under pressure in presence of base to get the product. In this patent, alkylation is carried out on gelled polymer, which was prepared in situ. After completion of alkylation, polymer was treated with methanol (4 times), 2M sodium chloride solution in water (7 times) and deionized water (6 times). Hence total 17 washings were given to the polymer on a pressure suction filter.

Thus there remains a need for a process for preparation of Colesevelam hydrochloride with desired chloride content. The present inventors thus developed a process for preparation of Colesevelam hydrochloride which consistently provides a product with desired chloride content and bromide content.

### OBJECT OF INVENTION

An object of the present invention is to provide pharmaceutical compositions comprising wet granulated bile acid sequestrant and process for their preparation.

Another object of the invention is to provide pharmaceutical compositions comprising wet granulated bile acid sequestrants and at least one pharmaceutical excipient, wherein the composition is free of reducing sugars.

Another object of the invention is to provide pharmaceutical compositions comprising wet granulated Colesevelam, wherein the composition comprises at least one polyol selected from the group consisting of inositol, sorbitol, mannitol, isomalt, xylitol, lactitol, erythritol and maltitol.

Another object of the invention is to provide a simple, industrially feasible and cost effective process for preparation of Colesevelam hydrochloride having consistency in chloride content and bile binding capacity.

Yet another object of the present invention is to provide Colesevelam hydrochloride with chloride content in the range of 3-8 meq/gm, preferably in the range of 4-8 meg/gm.

Yet another object of the present invention is to provide Colesevelam hydrochloride with bromide content not more than 0.5% w/w (0.06 meq/gm).

Another object of the present invention is to provide Colesevelam hydrochloride with bile binding capacity not less than 0.5 mM/gm, preferably not less than 2.30 mM/gm (by sodium Glycochenodeoxycholate hydrate method), not less than 0.5 mM/gm (by sodium Glycocholate hydrate method) and not less than 0.95 mM/gm (by sodium Taurodeoxycholate hydrate method).

### SUMMARY OF INVENTION

According to one aspect of the present invention, there is provided a pharmaceutical composition comprising wet granulated bile acid sequestrant and at least one pharmaceutical excipient; wherein the composition is free of reducing sugars. Bile acid sequestrant is selected from the group consisting of Colesevelam, Cholestyramine, Colestipol, Sevelamer and Colestimide.

According to another aspect, the bile acid sequestrant is an alkylated cross-linked polymer and the invention provides pharmaceutical compositions comprising wet granulated alkylated cross-linked polymer.

According to another aspect of the present invention, there is provided a pharmaceutical composition comprising wet granulated Colesevelam along with at least one pharmaceutically acceptable excipient, wherein the particles of Colesevelam are spherical or globular in shape.

According to another aspect of the present invention, there is provided a pharmaceutical composition comprising wet granulated Colesevelam having characteristics selected from group consisting of: bile binding capacity (BBC) not less than 0.5 mM/gm or chloride content in the range of 3.0 to 8.0 meq/gm or bromide content not more than 0.5 w/w or at least 50% particles having particle size less than 1000 microns.

According to another aspect of the present invention, there is provided a pharmaceutical composition comprising wet granulated Colesevelam or pharmaceutically acceptable salts thereof and at least one pharmaceutical excipient; wherein the composition comprises at least one polyol selected from the group consisting of inositol, sorbitol, mannitol, isomalt, xylitol, lactitol, erythritol and maltitol.

According to another aspect of the present invention, there is provided a combination of wet granulated bile acid sequestrant with an additional agent. The additional agent is selected from biguanide, sulfonyl urea, dipeptidyl peptidase IV inhibitor, thiazolidinedione, alpha-glucosidase inhibitor, meglitinide, fibrates and statins. Preferably, the additional agent is a biguanide such as metformin.

According to another aspect of the present invention, there is provided a process for preparation of wet granulated bile acid sequestrant comprising the steps of:

(a) providing the bile acid sequestrant;
(b) providing a wet-granulation solution said wet-granulation solution comprising at least about 70% (w/w) of organic solvent, said wet-granulation solution having not more than about 25% (w/w) of water;
(c) wet granulating the bile acid sequestrant with the wet-granulation solution to form granules and optionally formulating the resulting granules into final dosage form.

Preferably, the bile acid sequestrants are wet granulated by high shear granulation method or spray granulation method.

According to another aspect of the present invention, there is provided a process for preparation of wet granulated Colesevelam comprising the steps of:

(a) providing the Colesevelam;
(b) providing a wet-granulation solution said wet-granulation solution comprising at least about 70% (w/w) of organic solvent, said wet-granulation solution having not more than about 25% (w/w) of water;
(c) wet granulating the Colesevelam with the wet-granulation solution to form granules and optionally formulating the resulting granules into final dosage form.

According to another aspect of the present invention, there is provided a process for preparation of wet granulated pharmaceutical compositions of Colesevelam or pharmaceutically acceptable salts thereof comprising the steps of:

(1) providing a mixture of Colesevelam or pharmaceutically acceptable salts thereof and one or more pharmaceutically acceptable excipients;
(2) optionally wetting the mixture of step (1) using water or an aqueous solution of polyethylene glycol;
(3) providing a wet-granulation solution said wet-granulation solution comprising at least about 70% (w/w) of organic solvent, said wet-granulation solution having not more than about 25% (w/w) of water;
(4) granulating the mixture of step (1) or step (2) with wet granulation solution by high shear granulation or spray granulation to form granules;
(5) optionally compressing the granules into tablets or filling the granules into capsules.

According to one aspect of the present invention, there is provided a process for preparation of Colesevelam hydrochloride having chloride content in the range of 3.0 to 8.0 meq/gm and bromide content not more than 0.5 w/w comprising treating the alkylated crosslinked polymer with acid to get the product. Preferably, the product so obtained is dried in air tray dryer (ATD) or vacuum tray dryer (VTD) or fluidized bed dryer (FBD) or rotary evaporator for 1 to 48 hours at temperature of about 50-110° C. to get loss on drying (LOD) less than 10%.

According to another aspect of the present invention, there is provided a process for preparation of crosslinked polymer or salt thereof comprising treating crosslinked polymer or salts thereof with hydrochloric acid having concentration less than about 5N to get the product having chloride content in the range of 3-8 meq/gm preferably 4-8 meq/gm. Preferably, the crosslinked polymer is Sevelamer or Colesevelam.

According to another aspect of the present invention, there is provided a process for the preparation of Colesevelam hydrochloride comprising: a) alkylating cross linked polyallylamine polymer or salt thereof with 6-bromohexyltrimethylammonium bromide and 1-bromodecane in presence of alcoholic solvent and b) treating the obtained polymer with dilute HCl to get the product with desired chloride and bromide content.

Preferably, alkylation is carried out in the presence of a base selected from the group consisting of sodium hydroxide, potassium hydroxide, lithium hydroxide or mixtures thereof and at a temperature range of 25 to 90° C.

Preferably, alkylated crosslinked polymer obtained after alkylation is treated with sodium chloride solution or purified water to remove the inorganic matrix.

According to another aspect of the present invention, there is provided Colesevelam hydrochloride having characteristics selected from group consisting of: bile binding capacity (BBC) not less than 0.5 mM/gm or chloride content in the range of 3.0 to 8.0 meq/gm or bromide content not more than 0.5 w/w or at least 50% particles having particle size less than 1000 microns.

According to another aspect of the present invention, there is provided Colesevelam hydrochloride with bromide content not more than 0.5% w/w.

Preferably, Colesevelam hydrochloride obtained by the process of the present invention has a bile binding capacity (BBC) not less than 0.5 mM/gm when measured by method selected from Sodium Glycochenodeoxycholate hydrate method or Sodium Glycocholate hydrate method or Sodium Taurodeoxycholate hydrate method.

### BRIEF DESCRIPTION OF THE DRAWINGS

FIG. 1 shows spherical or globular shaped particles of Colesevelam hydrochloride (Active Pharmaceutical Ingredient) viewed through a microscope at a magnification of 40×.

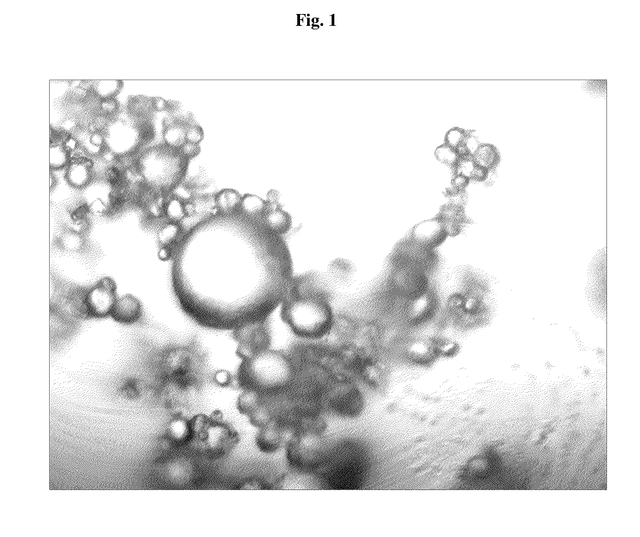

### DETAILED DESCRIPTION OF THE INVENTION

The present invention provides wet granulated bile acid sequestrant and at least one pharmaceutical excipient. The bile acid sequestrant is selected from the group consisting of Colesevelam, Cholestyramine, Colestipol, Colestimide or Sevelamer.

According to one embodiment, the invention provides pharmaceutical compositions comprising wet granulated alkylated cross-linked polymer.

According to one embodiment, the invention provides pharmaceutical compositions comprising wet granulated Colesevelam or pharmaceutically acceptable salt thereof. Particularly, the pharmaceutically acceptable salt is a hydrochloride salt of Colesevelam.

According to another embodiment, the present invention provides pharmaceutical compositions comprising wet granulated bile acid sequestrants substantially free of reducing sugars. Some reducing sugars are glucose, fructose, lactose, glyceraldehyde, arabinose and maltose. Reducing sugars in basic solution, forms aldehyde or ketone and allows the sugar to act as a reducing agent. Monosaccharides that contain ketone groups are known as ketoses and those which contain aldehyde groups are known as aldoses. Colesevelam contains the amine group which can undergo Maillard reaction in contact with excipients that contain reducing sugars. Maillard reaction is well known in the art and the products of maillard reaction are basically brown pigments. Formation of these brown pigments are indication of chemical instability of the composition.

According to another embodiment, the invention provides pharmaceutical composition comprising wet granulated bile acid sequestrant in combination with an additional agent. The additional agent is selected from biguanide, sulfonyl urea, dipeptidyl peptidase IV inhibitor, thiazolidinedione, alpha-glucosidase inhibitor, meglitinides, fibrates or statins.

The pharmaceutical compositions of the present invention comprises about 50.0% to about 85.0% by weight of Colesevelam or a pharmaceutically acceptable salt thereof. Further, the compositions of present invention comprises not more than about 90.0% by weight of hydrated bile acid sequestrant; particularly Colesevelam hydrochloride.

Colesevelam hydrochloride is water insoluble. However, it swells in contact with water and due to this tendency of swelling of Colesevelam hydrochloride, formulating Colesevelam hydrochloride by wet granulation becomes difficult. Prior art discloses various formulations of Colesevelam Hydrochloride by methods involving direct compression. The prior art thus teaches away from wet granulation. The swellable nature of Colesevelam when in contact with water provides a challenge to formulate Colesevelam by wet granulation. The inventors of the present invention tried out several ways for formulating Colesevelam hydrochloride and have successfully developed formulations by wet granulation.

According to another embodiment, the present invention provides a process for preparation of bile acid sequestrant compositions comprising wet granulation comprising the steps of:

(a) providing the bile acid sequestrant;
(b) providing a wet-granulation solution said wet-granulation solution comprising at least about 70% (w/w) of organic solvent, said wet-granulation solution having not more than about 25% (w/w) of water;
(c) granulating the bile acid sequestrant with wet-granulation solution to form granules and optionally formulating the resulting granules into final dosage form.

According to one embodiment, the invention provides a process for preparation of compositions of Colesevelam or pharmaceutically acceptable salts thereof; said process comprising wet granulation. Specifically, the invention describes a process for preparation of Colesevelam or pharmaceutically acceptable salts thereof comprising high shear granulation or spray granulation.

In the practice of the present invention, wet granulation is done by high shear granulation or spray granulation/fluid bed processing method. High shear granulation provides improved cohesiveness of particles, excellent flowability and compression characteristics. High shear granulation is performed using rapid mixer granulator or planetary mixer.

According to one embodiment, the particles of Colesevelam hydrochloride prepared according to the process as described herein are globular in shape, particularly spherical or round in shape. Spherical or round shaped particles have low bulk density and poor flow properties and further resist size reduction. Particles resist deformation and do not rupture or fracture. Due to these characteristics of Colesevelam hydrochloride, formulating Colesevelam hydrochloride by direct compression method becomes extremely difficult. In the practice of the present invention, the spherical morphology and hydrophilic nature of active ingredient Colesevelam hydrochloride presents a special challenge to the formulator.

According to another embodiment, the process for preparation of pharmaceutical compositions of Colesevelam or pharmaceutically acceptable salts thereof comprising wet granulation which comprises the steps of:

(1) providing a mixture of Colesevelam or pharmaceutically acceptable salts thereof and one or more pharmaceutically acceptable excipients;
(2) optionally wetting the mixture of step (1) using water or an aqueous solution of polyethylene glycol,
(3) providing a wet-granulation solution said wet-granulation solution comprising at least about 70% (w/w) of organic solvent, said wet-granulation solution having not more than about 25% (w/w) of water;
(4) granulating the mixture of step (1) or step (2) with wet granulation solution by high shear granulation or spray granulation to form granules;
(5) optionally compressing the granules into tablets or filling the granules into capsules.

According to a preferred embodiment, the process for preparation of compositions of Colesevelam or pharmaceutically acceptable salts thereof comprising wet granulation comprises the steps of:
(a) preparing a mixture of Colesevelam or pharmaceutically acceptable salts thereof and one or more diluents and optionally disintegrants;
(b) optionally wetting the mixture of step (a) using water or solution of polyethylene glycol;
(c) preparing a wet granulation solution by dissolving the binder in a mixture of organic solvent and water;
(d) granulating the mixture of step (a) or step (b) using wet granulation solution by high shear granulation or spray granulation to form granulated mass;
(e) drying the granulated mass;
(f) milling the dried granulated mass using ball mill or fluid energy mill to form granules of suitable size;
(g) lubricating the milled granules;
(h) compressing the lubricated granules into tablets or filling the lubricated granules into capsules;
(i) optionally coating the compressed tablets.

Colesevelam hydrochloride is not a free flowing powder and is bulky. Wetting with water helps in decreasing the interparticulate distance and increasing the contact area between the particles; thus making the Colesevelam hydrochloride more amenable for granulation. Alternatively, the mixture of active and diluent may be made wet using a solution of polyethylene glycol dissolved in water. In an alternate method, polyethylene glycol 6000 may be added into the dry mix as a fine powder during the mixing step. Polyethylene glycols of various grades may be used such as polyethylene glycol 6000 or the like. Wetting is carried out either in a rapid mixer granulator or a planetary mixer. In the practice of the present invention, wetting of mixture of Colesevelam and diluent is carried out using about 9% to 15% by weight of water.

In the practice of the present invention, the wet-granulation solution comprises at least about 70% (w/w) of organic solvent and not more than about 25% (w/w) of water. The organic solvent in said wet-granulation solution comprises lower-chain alcohol, selected from the group consisting of ethyl alcohol and isopropyl alcohol.

According to one embodiment of the invention, the process of preparation of compositions of Colesevelam hydrochloride by high shear granulation comprises providing a mixture of Colesevelam hydrochloride and one or more diluents and optionally disintegrants; granulating the dry mix by wet granulation method using mixture of a organic solvent and water and preferably by using a wet granulation solution prepared by dissolving the binder in a mixture of organic solvent and water; the granulation process being carried out in a rapid mixer granulator. Granulated mass is dried to remove the organic solvent and further dried at temperature of 50° C. to 60° C. till loss on drying value of about 8% to 12% is achieved. Dried granules are further milled or pulverized to get granules size less than 425 microns and preferably less than 150 microns using a multi-mill initially and then a fluid energy mill or a ball mill and preferably using a ball mill. Milled or pulverized granules are lubricated using lubricants known in the art and further compressed to provide tablets of required size. Compressed tablets are film coated by non-aqueous coating or aqueous coating or by hydro-alcoholic coating.

According to another embodiment, the process of preparation of compositions of Colesevelam hydrochloride by spray granulation comprises providing a mixture of Colesevelam hydrochloride and one or more diluents and optionally disintegrants; transferring the mixture into a fluid bed processor/spray granulator and granulating the dry mix by wet granulation method by spraying the wet granulation solution comprising a mixture of organic solvent and water and preferably using a wet granulation solution prepared by dissolving the binder in a mixture of organic solvent. The wet granulation solution is sprayed on the mixture until a granular wet mass is formed. The wet mass is air dried for sufficient time to remove the organic solvent and further dried at temperature of 50° C. to 60° C. till loss on drying of about 8% to 12% is achieved. Dried granules are further milled or pulverized to get granules of size less than 425 microns and preferably less than 150 microns using a multi-mill initially and then a fluid energy mill or a ball mill and preferably using a ball mill. Milled or pulverized granules are lubricated using lubricants known in the art and further compressed to provide tablets of required size. Compressed tablets are film coated by non-aqueous coating or aqueous coating or by hydro-alcoholic coating.

The spherical granules produced by the high shear granulation/spray granulation process may be filled along with suitable excipients into hard gelatin/cellulose capsules of suitable size. Capsule filling can be done using a suitable capsule filling machine. Compositions of the present invention may be in the form of granules, tablets or capsules.

According to one preferred embodiment, the process of preparation of Colesevelam hydrochloride compositions comprises mixing Colesevelam hydrochloride with one or more diluents and optionally disintegrants; wetting the mixture using a solution of polyethylene glycol 6000 (Macrogol) dissolved in water; preparing a wet granulation solution by dissolving polyvinyl pyrrolidone (Povidone K-30) in an organic solvent (isopropyl alcohol); granulating the wet mixture using said wet granulation solution and drying the granules. Sizing the dried granules through 100# on vibrosifter after milling with multi-mill and ball mill. The dried granules are blended using lubricants known in the art and the lubricated granules are further compressed into tablets. The core tablets are further film coated by aqueous coating process until a weight gain of 4.0% to 6.0% is achieved.

In the practice of the present invention, wet granulation is carried out by adding the hydro-alcoholic binder solution slowly in a thin stream continuously using a peristaltic pump under high speed mixing with the impeller ‘on’ and chopper ‘off’. On completion of addition of wet granulation solution/binder solution, mixing is continued at high impeller speed until cohesive granular mass is obtained. If the mass is lumpy then chopper may be used at high speed with impeller also at high speed to obtain uniform wet mass. If planetary mixer is used for granulation, the wet mass is to be milled on a multi-mill using 8.0 mm screen and then charged for drying.

In the practice of the present invention, high shear granulation improves the cohesiveness of particles and provides excellent flowability and compression characteristics to the tablet. As the granules exhibit good flow properties, the tablets produced possess uniformity in weight. Drying of wet mass may be carried out using fluidized bed drier or tray drier. Initial drying is performed without application of temperature so as to remove the organic solvent and further the wet mass is dried for sufficient time at about 45° C. to 50° C. until loss on drying value is achieved in the range of about 5.0% to about 12.0%.

According to another embodiment of the invention, the process of preparation of Colesevelam hydrochloride composition comprises providing a mixture of active ingredient Colesevelam hydrochloride and one or more excipients; granulating the mixture by high shear granulation using a organic solvent and preferably by using a wet granulation solution prepared by dissolving the binder in organic solvent; the granulation process being carried out in a rapid mixer granulator. Granulated mass is further dried to remove the organic solvent and further dried until a loss on drying value in the range of about 5.0% to 12.0% (which is similar to moisture content of active Colesevelam hydrochloride) is achieved. Dried granules are further milled or pulverized to get granules size of about 425 microns and preferably less than 150 microns. Milling may be carried out using a fluid energy mill or a ball mill and preferably by using a ball mill. Milled or pulverized granules are lubricated using lubricants known in the art and further compressed to provide tablets of required size or filled into capsules. Compressed tablets may be further coated.

According to another embodiment, the process comprises mixing Colesevelam hydrochloride with one or more diluents and optionally disintegrants; optionally wetting the mixture using water in a rapid mixer granulator; preparing a wet granulation solution by dissolving ethyl cellulose in an organic solvent such as isopropyl alcohol; granulating the mixture of Colesevelam hydrochloride and diluents using said wet granulation solution and drying the granules. Sizing the dried granules through 100# on vibrosifter after milling with multi-mill and ball mill and further blending with commonly used lubricants and compressing the granules. Core tablets are further film coated.

According to another embodiment, Colesevelam hydrochloride is mixed with mannitol and made wet using water; granulated using wet granulation solution prepared by dissolving the ethyl cellulose in isopropyl alcohol. Granulation is carried out in a rapid mixer granulator and the granulated mass is dried to remove the organic solvent until loss on drying of about 5.0% to about 12.0% is achieved. Dried mass is sized using ball mill to achieve granules of required size; lubricated using lubricants and compressed into tablets.

The granules provided by high shear granulation process as described herein are spherical granules of size less than about 425 microns and preferably less than 150 microns. Although the dried granulated mass can be milled or pulverized using conventional equipments known in the art such as a multimill, co-mill, cadmill or fitzmill, they have limitations when used for size reduction of Colesevelam hydrochloride granules. Granule size below 425 microns (which passes through 40#) is difficult to obtain using such mills. Large granules pose difficulties during compression by decreasing the compressibility of the granules and produces porous tablets with low hardness, which consequently exhibit high friability and pose a risk of moisture uptake during aqueous film coating. Oversized granules retained after milling through 0.5 mm screen on a conventional mill and sifting on a vibrosifter through 100# are milled in a ball mill or fluidized energy mill to obtain granules having particle size below 425 microns and preferably below 150 microns.

Size reduction or pulverization using fluid energy mill or ball mill provides spherical granules of size less than 425 microns, which provides ease in compressibility. Ball milling being the preferred mode for size reduction of granules. Ball mill is preferred in terms of output and productivity for large scale batches. In ball milling, the process of size reduction occurs due to combined effect of impact and attrition. In a Fluid energy mill, the material is suspended and conveyed at high velocity by air, which is passed through nozzles at 100 to 150 pounds per square inch. The violent turbulence of the air reduces the particle size by inter-particulate attrition.

Milled mass is further sifted through a vibrosifter and oversized particles are milled through a mill preferably a ball mill with stainless steel balls and further sifted through a vibrosifter. The grinding media in the ball mill could range from Ytrria stabilized Zirconia systems, Glass, Porcelain, stainless steel, and the like. Mass is repeatedly milled with ball mill and sifted through vibrosifter until the resultant granules passes through 100#.

According to a preferred aspect, granules of the present invention preferably have a particle size of 100% passing through 100#. Particles of size 150 microns or less provide satisfactory compression of granules and further provides elegant non-porous, non-friable tablets with a smooth impervious surface, which can withstand the rigors of aqueous film coating. In the practice of the present invention, the granule size is controlled and 100% granules passes through 100# and provides tablets which exhibit a smooth impervious surface with a high hardness, low friability of less than 1.0% and preferably in the range of 0 to 0.8%, low disintegration time and a smooth aqueous film coating operation. By controlling the granule size at less than 425 microns (which passes through 40#) and preferably less than 150 microns (which passes through 100#), elegant tablets are produced.

Tablets may be compressed using suitable punches and dies. Tablets may be of oval, elliptical, spherical or caplet shape. Compression can be carried out using equipments known in the art such as a rotary tablet press. Tablets prepared by the process according to the invention meet the specification for disintegration (Limit not more than 30 minutes). Other parameters of tablets such as hardness, friability, and thickness, were measured and the results meet the standard specifications for tablets.

According to another embodiment of the invention, Colesevelam hydrochloride tablets may be coated by aqueous or non-aqueous or hydroalcoholic coating. Film coating provides an impervious surface and prevents the ingress of moisture from the aqueous coat.

In one embodiment, coating of tablets is done using an aqueous coating method. Aqueous coating of hydrophilic active ingredient is another difficult process and posed a real challenge to the inventors of the present invention as the Colesevelam hydrochloride has a tendency to swell in presence of water. Aqueous coating has been achieved by having a fine control on the hardness of the cores, which balances the need for a hard core to ensure good coating as well as meets the requirement for disintegration of coated tablets. As the tablet core is hard with an impervious smooth surface, it withstands the aqueous film coating and the polymer Colesevelam hydrochloride does not swell during coating.

According to one embodiment, the compositions of the present invention comprises the active ingredient Colesevelam hydrochloride in the range of about 50% to about 85% by weight of total composition. More particularly, Colesevelam hydrochloride compositions of the present invention may be provided in dose strength of 625 mg. Colestipol and cholestyramine may be provided in high doses of about 1000 mg.

According to one aspect of the present invention, the composition contains about 50.0% to about 85.0% by weight of Colesevelam hydrochloride, 5.0% to 20.0% by weight of diluent, 5.0% to 30.0% by weight of binder, 0.25% to 5.0% by weight of glidant, 0.25% to 5.0% by weight of lubricants and about 3.8 to 6.0% by weight of coating agents.

In the practice of the present invention, the bile acid sequestrant is selected from the group consisting of cholestyramine, colestipol, sevelamer, Colesevelam or colestimide. Colestipol and Cholestyramine are basic anion exchange resin; hydrophilic but insoluble in water. They swell in aqueous fluids and hence pose the same challenge in formulation by wet granulation. The features of the present invention can be extended to these other bile acid sequestrants as well.

According to another embodiment, the process of preparation of pharmaceutical compositions comprising wet granulated Colestipol Hydrochloride comprises the steps of mixing colestipol and one or more diluents, optionally wetting the mixture using water, preparing the wet granulation solution by dissolving ethylcellulose in ethyl alcohol and granulating the mixture with the said wet granulation solution in a rapid mixer granulator by high shear granulation method to form a highly cohesive mass. The wet mass is air dried to remove the organic solvent and further dried in a fluid bed drier at 50° C. to 60° C. The dried granules may further be sifted through appropriate fine mesh of about 100# and the oversized granules may be milled in ball mill with further sizing through the same mesh. These granules may further be compressed with addition of glidant and lubricant to produce elegant tablets. The core tablets may further be film coated. The coated tablets remain intact during swallowing but readily disintegrate in the stomach and the film coat maintains its integrity under accelerated conditions over extended shelf life period.

The pharmaceutical compositions of the present invention, particularly the tablet dosage form has a hardness of at least about 50 Newtons. Preferably, the tablets have a hardness in the range of about 50N to 200N [Hardness as measured in Newtons (N)].

According to one embodiment, the present invention provides pharmaceutical composition comprising wet granulated Colesevelam where at least 50% particles of Colesevelam has particle size less than 1000 microns.

According to another embodiment, the present invention provides pharmaceutical composition comprising wet granulated Colesevelam having characteristics selected from group consisting of: bile binding capacity (BBC) not less than 0.5 mM/gm when measured by method selected from Sodium Glycochenodeoxycholate hydrate method or Sodium Glycocholate hydrate method or Sodium Taurodeoxycholate hydrate method.

Table 1 shows the comparative analysis of bile binding capacity for Colesevelam hydrochloride tablets WELCHOL® and tablets prepared by the process of the present invention.

TABLE 1

| | | | Colesevelam |
|---|---|---|---|
| | | | hydrochloride |
| S. | | | tablets of |
| No. | Parameters | WELCHOL ® | present invention |
| 1 | BBC (mM/gm) by sodium | 3.88 | 4.24 |
| | Glycochenodeoxycholate | | |
| | hydrate method | | |
| 2 | BBC (mM/gm) by sodium | 1.30 | 1.10 |
| | Glycocholate hydrate method | | |
| 3 | BBC (mM/gm) by sodium | 1.72 | 1.78 |
| | Taurodeoxycholate hydrate | | |
| | method | | |

Compositions of present invention may include one or more pharmaceutically acceptable excipients selected from diluents, disintegrants, binders, lubricants, glidants, colorants, coating agents, plasticizers and the like.

Diluents are substances which provides bulk to the composition. Suitable diluents for use in the pharmaceutical composition of the invention include, but are not limited to maize starch, microcrystalline cellulose of various grades like Avicel PH 101, 112, 102, pregelatinized starch, calcium carbonate, calcium sulfate, polyols selected from the group consisting of inositol, sorbitol, mannitol, isomalt, xylitol, lactitol, erythritol or maltitol and the like. Excipients such as mannitol and isomalt have an advantage of low moisture pick up and satisfactory flow characteristics. Most preferred diluent for use in the pharmaceutical composition of the present invention is mannitol.

Diluents may be used in the range of about 5% to about 20% by weight of total composition. Considering the end use of the formulation, mannitol is the preferred diluent. On oral administration, mannitol is not absorbed significantly from the gastrointestinal tract. Granular and spray dried forms of mannitol are generally used in granulations. Granules containing mannitol get easily dried and they have less tendency to pick up moisture. Colesevelam hydrochloride being hygroscopic, mannitol is the preferred diluent as it is not hygroscopic. Various grades of mannitol are available commercially. Preferred grades of mannitol include Pearlitol SD 200 of Roquette, France.

Isomalt is hydrogenated isomaltulose which is an equimolar mixture of 6-O-α-D-glucopyranosido-D-sorbitol (1,6-GPS) and 1-O-α-D-glucopyranosido-D-mannitol-dihydrate (1,1-GPM-dihydrate). Isomalt is a non carcinogenic excipient commonly used in pharmaceutical preparations. Isomalt is available in various grades based on the particle size where fine grade viz. GalenIQ 810 is used in the wet granulation. Isomalt is preferably used in the range of about 0.2% to about 7%.

Disintegrants are agents added to tablet formulations to promote the breakup of the dosage form into smaller fragments in an aqueous environment thereby increasing the available surface area and promoting a more rapid release of the drug substance. Disintegrants include, but are not limited to modified starches viz. sodium starch glycollate (EXPLOTAB®, PRIMOJEL®), crosslinked polyvinyl pyrrolidones (Polyplasdone XL, Kollidon CL), modified celluloses viz. sodium carboxymethyl cellulose, calcium carboxymethyl cellulose (Ac-Di-Sol, Nymcel). Disintegrants may be used individually or in combination thereof. The preferred disintegrant is croscarmellose sodium.

Binders impart cohesiveness to tablet formulation and ensures that the tablet remain intact after compression. Binders which may be used according to the invention include, but are not limited to hydroxy propyl methyl cellulose, hydroxy propyl cellulose, hydroxy ethyl cellulose, ethyl cellulose, cellulose derivatives, maize starch, polyvinylpyrrolidone alone or in combination with polyethylene glycols and the like. Binders may be used in the range of about 5% to about 30% by weight of total composition. Preferred binder being ethyl cellulose and polyvinyl pyrrolidone. Different grades of ethyl cellulose having various viscosities are commercially available. Ethyl cellulose of specific grades or blends of different grades may be used to obtain solutions of desired viscosity. Ethyl cellulose having viscosity in the range of 4 cps to 22 cps is used; preferred being ethylcellulose with viscosity of about 5 to 15 cps. The preferred grade of ethylcellulose used for Colesevelam hydrochloride tablets is Ethocel EC-N 7 Pharm manufactured by Dow chemical company. Ethylcellulose is not metabolized following oral consumption and therefore a non-calorific substance. One or more binders may be used for preparing the wet granulation solution.

Solvents that may be used as per the invention include, isopropyl alcohol, ethyl alcohol, dichloromethane, water or mixtures thereof. Preferred solvent being isopropyl alcohol alone or a mixture of isopropyl alcohol and water or ethyl alcohol alone or ethyl alcohol/water mixture. The organic solvent/water solution is a solution of about 70% to 100% organic solvent and about 0% to 25% water.

Lubricants that may be used as per the invention include, but are not limited to stearic acid, calcium stearate, glyceryl monostearate, glyceryl palmitostearate, zinc stearate, magnesium stearate, sodium stearyl fumarate, calcium stearyl fumarate, hydrogenated vegetable oil, mineral oil, polyethylene glycol, sodium lauryl sulphate and the like. Glidants which may be used include colloidal silicon dioxide, talc and the like. Lubricants and glidants may be used in the range of about 0.25% to about 5% by weight of total composition.

Film coating may be carried out using polymers such as polyvinyl alcohol, hydroxyethyl cellulose, ethyl cellulose, hydroxypropyl methyl cellulose and methacrylic acid co-polymers. Ready mix coating materials may comprise plasticizers selected from propylene glycol, triacetin or polyethylene glycol. Coating agents may be used in the range of about 3.0% to about 6.0% by weight of total composition.

Composition prepared by the process as described herein has a water content of about 5.0% to about 12%, particularly about 7.0% to about 10.0%. Although the present invention makes use of organic solvents such as isopropyl alcohol or ethyl alcohol for granulation, the organic volatile impurity level in the finished product is quite low and is within the permissible limit. (Limit as per ICH guidelines: 5000 ppm)

Compositions of Colesevelam hydrochloride, particularly the tablets may be packed in aluminium strips or by cold formed blister pack, which is a cold process of blister packing, which acts as an excellent moisture barrier with negligible moisture vapor transmission rate and adequate environmental protection during shelf life. Alternatively, the bulk pack in HDPE containers also should suffice to maintain the quality and integrity.

Pharmaceutical compositions of the present invention shows an excellent chemical and physical stability. Compositions does not undergo discoloration due to Maillard reaction. Compositions prepared by the novel process as described herein withstand the accelerated stability conditions of temperature and relative humidity and maintain their physical and chemical integrity at accelerated conditions of stability.

Pharmaceutical composition comprising wet granulated Colesevelam is useful as an adjunct to diet and exercise to reduce elevated low-density lipoprotein cholesterol in patients with primary hyperlipidemia as monotherapy or in combination with an hydroxymethyl-glutaryl-coenzyme A (HMG CoA) reductase inhibitor.

Wet granulated Colesevelam according to the present invention can also be used as an adjunct to diet and exercise to improve glycemic control in adults with type 2 diabetes mellitus. The pharmaceutical composition comprising wet granulated Colesevelam can also be provided in combination with an additional agent. The additional agent is selected from biguanide, sulfonyl urea, thiazolidinedione, dipeptidyl peptidase IV inhibitor, alpha-glucosidase inhibitor, meglitinides, fibrates and statins. Preferably, the additional agent is biguanide. Biguanide is selected from the group consisting of metformin, buformin, phenformin and pharmaceutically acceptable salts thereof; sulfonylurea is selected from the group consisting of gliclazide, glipizide, glyburide and glimeperide; thiazolidinedione is selected from the group consisting of troglitazone, pioglitazone and rosiglitazone; dipeptidyl peptidase IV inhibitor is selected from the group consisting of sitagliptin, vildagliptin, saxagliptin and alogliptin; alpha.-glucosidase inhibitors selected from the group consisting of miglitol, acarbose and voglibose; meglitinide selected from the group consisting of repaglinide and nateglinide; fibrate selected from the group consisting of fenofibrate, gemfibrozil, bezafibrate, clofibrate and ciprofibrate; statin is selected from the group consisting of simvastatin, lovastatin, fluvastatin, atorvastatin, cerivastatin, pravastatin, rosuvastatin and mixtures thereof.

According to one embodiment, the present invention provides a method of treating a patient suffering from hyperlipidemia comprising administering to the patient the wet granulated Colesevelam hydrochloride pharmaceutical composition comprising a therapeutically effective amount of Colesevelam hydrochloride. Compositions of the present invention are safe for administration to diabetic patients.

The pharmaceutical composition of present invention comprising wet granulated Colesevelam may also be provided in combination with metformin or pharmaceutically acceptable salt thereof. Metformin or its pharmaceutically acceptable salt may be present in an amount from 250 mg to 2000 mg and Colesevelam or its pharmaceutically acceptable salt may be present in an amount from 500 mg to 4000 mg. Metformin hydrochloride is provided in the dose of 850 mg twice a day and Colesevelam Hydrochloride is provided in a dose of 3.75 gm/day.

As used herein, the term “therapeutically effective amount” refers to an amount sufficient to cause an improvement in a clinically significant condition in the patient or even prevent a disease, disorder or condition in a patient.

As used herein, the term “composition”, unless otherwise defined refers to granules and/or solid oral pharmaceutical dosage forms of bile acid sequestrants as disclosed in the present invention.

As used herein, the term “excipient” refers to pharmaceutically acceptable ingredient that is commonly used in the pharmaceutical technology for preparing pharmaceutical dosage forms.

As used herein, the term “tablet” is intended to encompass compressed pharmaceutical dosage forms of all shapes and sizes, whether coated or uncoated.

According to one embodiment, the present invention describes process for the preparation of Colesevelam hydrochloride having chloride content in the range of 3-8 meq/gm, preferably 4-8 meq/gm, most preferably 4.77-7.15 meq/gm, bromide content not more than about 0.5% w/w and bile binding capacity not less than 0.5 mM/gm, preferably not less than 2.30 mM/gm (by sodium Glycochenodeoxycholate hydrate method) or not less than 0.5 mM/gm (by sodium Glycocholate hydrate method) or not less than 0.95 mM/gm (by sodium Taurodeoxycholate hydrate method), most preferably 3.2-4.8 mM/gm.

According to the present invention, process for preparation of Colesevelam hydrochloride comprises the following steps,
1. reacting 1,6-dibromohexane and trimethylamine at low temperature to get 6-bromohexyl trimethylammonium bromide;
2. alkylating Sevelamer hydrochloride using 6-bromohexyl trimethylammonium bromide obtained in step 1) and 1-bromodecane in presence of base to get the bromide salt of Colesevelam;
3. converting the obtained bromide salt into chloride salt using sodium chloride solution;
4. treating the obtained polymer with acid and
5. isolating Colesevelam hydrochloride.

The process for preparation of Colesevelam hydrochloride is performed in two stages.

### Stage 1:

Process for preparation of 6-bromohexyltrimethylammonium bromide (Quaternisation) comprises reaction of 1,6-dibromohexane with trimethylamine in suitable organic solvent at low temperature. The separated product is filtered in nitrogen atmosphere since product formed is hygroscopic in nature. The product is dried under vacuum at 25 to 60° C., preferably 40 to 45° C. The second crop of the product is obtained by stirring the mother liquor for several hours. The reaction is represented in the following reaction scheme:

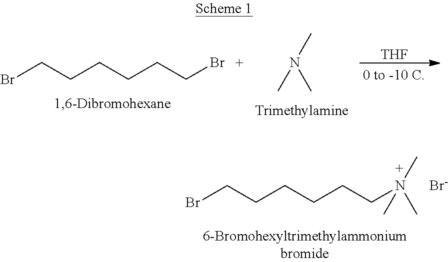

The obtained 6-bromohexyltrimethylammonium bromide (bromide content 25.6%) is used as such for alkylation of polyallylamine hydrochloride. The low temperature is in the range of −10° C. to 30° C., preferably −10° C. to 0° C.

The solvent used is selected from ether, hydrocarbon, ester, ketone, preferably ether, more preferably tetrahydrofuran. The reaction is carried out at the temperature range of −10° C. to 30° C., preferably −10° C. to 0° C. for 12 to 60 hours, preferably 48 hours.

Mole ratio of 1,6-dibromohexane to trimethylamine is in the range of 1:0.9 to 1:1.2, preferably 1:1.06.

### Stage 2:

Process for preparation of Colesevelam hydrochloride comprises partially neutralising Sevelamer hydrochloride using base in organic solvent and adding alkylating agents to the obtained reaction mass. The reaction mass is refluxed at 60-65° C. for several hours, optionally under pressure. After the completion of reaction, the obtained material is filtered, washed with organic solvent by vigorous stirring followed by two to three times washing with sodium chloride solution to get complete bromide exchange with chloride ions. The polymer thus obtained is treated with acid to get the product with desired chloride content. The product is washed two to three times with purified water to remove any inorganic impurities and dried either on rotary evaporator or fluidized bed dryer at an elevated temperature preferably at 40-90° C. for 8 to 24 hrs.

Said alkylation is carried out at temperature in the range of 25 to 90° C. The alkylated polymer is washed with sodium chloride solution or purified water to remove the inorganic matrix. The sodium chloride solution is in the range of 0.1 to 10 M in purified water.

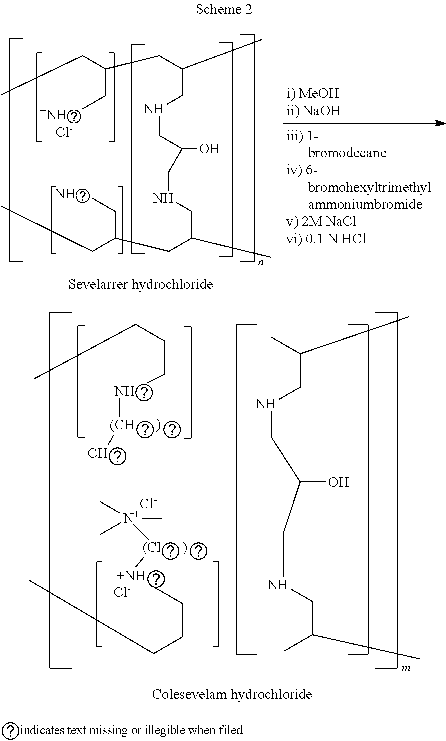

The product obtained is dried in air tray dryer (ATD) or vacuum tray dryer (VTD) or fluidized bed dryer (FBD) or rotary evaporator for 1 to 48 hours at temperature of about 50-110° C. to maintain loss on drying content less than 10%.

The base used for alkylation is selected from alkali or alkaline earth metal hydroxides, preferably sodium hydroxide.

The organic solvent used for the reaction is selected from alcohols such as methanol, ethanol, n-propanol, isopropanol, preferably methanol.

The alkylating agents used are 6-bromohexyl trimethylammonium bromide and 1-bromodecane.

The acid used is hydrochloric acid. The alkylated polymer is treated with acid preferably hydrochloric acid having concentration less than 5 N, preferably 0.1 N solution in purified water. The acid treatment may be repeated 2-5 times.

According to one preferred embodiment, poly(allylamine hydrochloride) and water are mixed at 25 to 35° C. to get a clear solution. The solution is further cooled to 5 to 15° C. and sodium hydroxide solution in water (65-70% of sodium hydroxide pellets per mole of polyallylamine hydrochloride) is added to the reaction mass at 5 to 15° C. and stirred for 30 minutes. Toluene and SPAN-85 are added to it at 5 to 15° C. The temperature of the reaction mixture is then raised to 20 to 25° C. and maintained for 15 min. The reaction mixture is filtered to remove any extraneous matter at 25 to 35° C. The temperature of the filtrate is further raised to 55 to 60° C. and maintained for 15 minutes. Epichlorohydrin (5-12% by wt of Polyallylamine hydrochloride) is added at constant temperature of 55 to 60° C. to reaction mixture and maintained for 3 hr at 55 to 60° C. The reaction mixture is cooled to 25 to 35° C. and product is isolated by centrifugation. The wet cake is further sludged with water for 45 min at 25 to 50° C., filtered and dried in FBD at 25 to 90° C. to get crosslinked polyallylamine polymer.

According to another preferred embodiment, 1,6-dibromohexane is stirred in suitable solvent such as tetrahydrofuran (THF) and trimethylamine is slowly purged into reaction mass at −10 to 0° C. for 2-3 hrs. The reaction mass is further stirred for 48 hrs and the separated solid is immediately filtered, washed with tetrahydrofuran, suck dried and then dried in vacuum oven at 40-45° C. to get 6-bromohexyltrimethylammonium bromide in good yield.

Another embodiment of the present invention provides process for preparation of Colesevelam salts comprising reaction of cross linked polyallylamine polymer or salt thereof with base such as alkali earth metals hydroxides to make amine sites of polymer free for alkylation and alkylation of the obtained polymer with 6-bromohexyltrimethylammonium bromide and 1-bromodecane in presence of alcoholic solvent selected from the group of methanol, ethanol, n-propanol, isopropanol, n-butanol and like or mixtures thereof. The wet cake obtained is optionally stirred in sodium chloride solution in water for 2 hrs twice and filtered and further stirred in purified water for 1 hr twice to get desired product.

The base used is alkali earth metals hydroxides selected from the group consisting of sodium hydroxide potassium hydroxide, lithium hydroxide or mixtures thereof.

The alcoholic solvent is selected from the group consisting of methanol, ethanol, n-propanol, isopropanol, n-butanol and the like or mixtures thereof.

The alkylation is carried out at temperature in the range of 25 to 90° C.

The alkylated polymer is preferably washed with sodium chloride solution or purified water to remove the inorganic matrix.

The sodium chloride solution used is in the range of 0.1 to 10 M in purified water.

According to the preferred embodiment of the present invention, the alkylation reaction of crosslinked polyallylamine such as Sevelamer hydrochloride with 6-bromohexyl trimethylammonium bromide and 1-bromodecane is carried out under pressure and after the completion of alkylation reaction the product obtained is filtered either using Buchner funnel assembly or by centrifuging. The obtained wet cake is then washed with alcoholic solvent and filtered either using Buchner funnel assembly or by centrifuging. The bromide counterion exchange with chloride ion is carried out by vigorous stirring of wet cake in aqueous sodium chloride solution, optionally one or more times at temperature from 10° C.-40° C. preferably at 25° C.-35° C. The obtained wet cake is treated with aqueous hydrochloric acid solution, preferably 0.1N hydrochloric acid, at temperature from 10° C.-40° C. preferably at 25° C.-35° C. to get the product with desired chloride content. The wet cake is further stirred vigorously preferably, one or more times, in purified water at temperature from 10° C.-40° C. preferably at 25° C.-35° C. to remove inorganic impurities in the desired product. The obtained wet cake is dried on rotary evaporator or fluidized bed dryer at an elevated temperature of 60° C. to 95° C. to get the material in high yield.

Another embodiment of the present invention provides process for preparation of cross linked polymer salts which comprises treating the alkylated polymer with acid to get the product with desired chloride content and bromide content as compared to the prior art processes.

According to preferred embodiment of the present invention, the treatment of alkylated polymer with acid surprisingly results in the product with desired chloride and bromide content as regards prior art where there is no discussion about the chloride content and bromide content of Colesevelam hydrochloride. The amount of these contents in the product depend upon the conditions and important parameters of process which are described and discussed in the present application. Comparative analysis of Colesevelam HCl prepared according to the present invention, prior art process (U.S. Pat. No. 5,693,675) and WELCHOL® tablet is as follows,

| Sr. | | WELCHOL ™ | U.S. Pat. No. | Present |
|---|---|---|---|---|
| No | Parameters | tablet | 5,693,675 | invention |
| 01 | BBC (mM/gm) | 4.0 | 3.07 | 4.12 |
| 02 | Chloride (meq/gm) | 5.96 | 2.03 | 5.45 |
| 03 | Bromide (meq/gm) | 0.09 | 0.35 | Less than |
| | | | | about 0.1 |

The process for present invention is efficient and consistent to provide Colesevelam salt having the bromide content not more than 0.5% w/w (0.06 meq/gm) to ensure that an API meets the specifications for its intended use whereas the innovator's marketed formulation, WELCHOL®, consists of 0.75% (w/w) bromide content. The comparative results of the batches taken by following the process of the present invention and WELCHOL®, are tabulated as below,

| Sr. | | Bromide content |
|---|---|---|
| No. | Batch No. | % w/w (meq/gm) |
| 1 | CS/RL/0712044 | 0.3 (0.04 meq/gm) |
| 2 | CS/RL/0712045 | 0.26 (0.03 meq/gm) |
| 3 | CS/RL/0712046 | 0.22 (0.03 meq/gm) |
| 4 | CS/RL/0712047 | 0.13 (0.02 meq/gm) |
| 5 | CS/RL/0712048 | 0.17 (0.02 meq/gm) |
| 6 | WELCHOL ®, | 0.75 (0.09 meq/gm) |
| | tablets | |

Generally, salt formers are classified in three classes,

Class I are those which can be used without restriction because they form physiologically ubiquitous ion or they occur as intermediate metabolites in biochemical pathways. e.g., Hydrochlorides, Chlorides and Sodium salts.

Class II are salt formers which are not occurring naturally, but their use has shown low toxicity and good tolerability.

Class III salt formers are introduced in particular situation in order to achieve special effect or for solving particular problems. Salt formers of this class have been less frequently used in past. Class III is made up of candidates, whose safety status is known to be somewhat less desirable, and hence viewed as less advisable.

The criteria for selection of anion or cation for formation of salt is closely related to natural abundance of the ion under physiological condition. Chloride and phosphate are abundant anionic constituents of the body and for this reason, are tolerated best. Chloride holds the first place amongst the exchangeable anions of the body. Bromides should be avoided for salt formation, since they can accumulate in so called chloride environment. Bromism is characterized by acne and furunculosis, followed by central-nervous-system symptoms such as drowsiness. The hydrochloride salt of a drug has more bioavailability compared to different salts. Hydrochloric acid is regarded as class I where as Hydrobromic acid is regarded as Class III. The maximum daily dose of Colesevelam is about 3.75 g. Hence unwanted bromide must be controlled as low as possible to make the API safe for human consumption. (Handbook of Pharmaceutical salts: Properties, Selection and Use. P. Heinrich stahl, Camille G Wermuth. Wiley-VCH, 2002, Page 117-129, 329-345)

Advantages of the present invention:

1. The process of the present invention provides Colesevelam hydrochloride in a yield which is higher (about 200%, whereas the process of U.S. Pat. No. 5,693,675 provides about 150%).
2. The polymer obtained by the process of prior art (U.S. Pat. No. 7,148,319 and U.S. Pat. No. 7,105,631) was washed more than 10 times whereas in the present invention less number of washings are required (i.e. 7 times) to yield the product with desired chloride content, bile binding capacity and bromide content.
3. The process of the present invention provides Colesevelam salt with desired chloride content and bile binding capacity.
4. The process of the present invention is an efficient, consistent, non-tedious, non-laborious, cost effective, commercially viable process as Sevelamer hydrochloride used in the present process is the dry powder instead of gelled polymer as used in prior art.

According to another embodiment of the present invention there is provided Crosslinked polymer or salts thereof having bile binding capacity (BBC) not less than 0.5 mM/gm, preferably not less than 2.30 mM/gm (by sodium Glycochenodeoxycholate hydrate method), not less than 0.5 mM/gm (by sodium Glycocholate hydrate method) and not less than 0.95 mM/gm (by sodium Taurodeoxycholate hydrate method), most preferably in the range of 3.2-4.8 mM/gm. The methods used for determining the bile binding capacity are disclosed in J. of Pharm. Sci. 95, 12, 2751-2759.

The term “room temperature” refers to about 20° C. to 45° C. and all temperatures and temperature ranges herein refer to degrees Centigrade.

The term “inorganic matrix” refers to residue on ignition as understood by a person skilled in the art.

Another embodiment of the present invention provides Colesevelam hydrochloride with chloride content in the range of 3.0 to 8.0 meq/gm, preferably in the range of 4.0-7.0 meq/gm, more preferably 4.77-7.15 meq/gm.

Another embodiment of the present invention provides Colesevelam hydrochloride with bromide content not more than 0.5% w/w.

Colesevelam hydrochloride obtained according to the process of the present invention may have particle size distribution such that 50 vol % or greater of Colesevelam particles have particle size less than 1000 microns, which includes 75 vol % or greater, 85 vol % or greater, 90 vol % or greater or 100 vol % of Colesevelam hydrochloride particles having particle size less than 1000 microns. Preferably 90% or greater than 90% particles have particle size in the range of about 100 to 500 microns, more preferably in the range of about 100-300 microns. The particle size may be further reduced by conventional techniques such as micronization, microfludization, milling etc. to increase the surface area of the active. The micronized particles may have 90% or greater than 90% of the particles having volume diameter less than 100 microns.

According to another embodiment Colesevelam hydrochloride may have particle size distribution such that at least 50% of the particles have particle size less than 1000 microns, preferably at least 75% of the particles have particle size less than 1000 microns, more preferably at least 90% of the particles have particle size less than 1000 microns. Preferably at least 90% particles have particle size in the range of about 100 to 500 microns, more preferably in the range of about 100-300 microns.

Still another embodiment of the present invention provides Colesevelam hydrochloride with bromide content not more than 0.5 w/w (0.06 meq/gm).

According to one embodiment of the present invention, the wet granulated composition of Colesevelam hydrochloride is formulated using the active ingredient Colesevelam hydrochloride prepared by the process as described herein or any other alternate process.

The present invention is further illustrated by reference to the following examples which does not limit the scope of the invention in any way. It will be apparent to those skilled in the art that many modifications, both to the materials and methods, can be practiced without departing from the purpose and scope of the disclosure.

### EXAMPLES

### Example 1

| Ingredients | Quantity (g) |
|---|---|
| Core | |
| Colesevelam hydrochloride | 156.250 |
| Mannitol | 20.400 |
| Polyvinyl pyrrolidone (Povidone 30) | 22.500 |
| Purified water | 17.550 |
| Isopropyl alcohol | 62.500 |
| Colloidal silicon dioxide | 2.200 |
| Magnesium stearate | 1.100 |
| Film-coating | |
| Ready mix film coating material | 8.800 |
| Total | 228.800 |

Procedure: Colesevelam hydrochloride was co-sifted with mannitol (Pearlitol SD 200) using 20# stainless steel sieve and transferred into a rapid mixer granulator and mixed for 5 minutes at 100 rpm. Wet granulation solution was prepared by dissolving povidone in a mixture of isopropyl alcohol and water. Wet granulation solution was added to the Colesevelam-mannitol mixture and mixed at impeller high speed 180 to 200 rpm with chopper off condition for sufficient time till a cohesive mass was formed. The mass was air dried for sufficient time in Glatt drier and further dried at temperature of 50° C. to 60° C. till loss on drying value of about 8% to 12% was achieved. The dried granules were sifted through 100# Sieve. The over sized granules were milled using ball mill and the milled mass was sifted through 100# sieve. The sifted granules were blended with presifted colloidal silicon dioxide (sifted through 40#) and magnesium stearate (sifted through 60#) in a conta blender and compressed on 0.75×0.35 inch elliptical punches and dies to obtain tablet having weight of 880 mg per tablet and hardness of about 80 to 200 N. The compressed tablets were coated using coating solution prepared by dissolving coating ready mix containing HPMC 5 cps and Triacetin in water, till weight gain of 4.0% of the core tablets (weight of coated tablets 915.200 mg) was achieved.

### Example 2

| Ingredients | Quantity (g) |
|---|---|
| Core | |
| Colesevelam hydrochloride | 625.000 |
| Mannitol | 81.600 |
| Polyvinyl pyrrolidone (Povidone 30) | 90.000 |
| Purified water | 70.200 |
| Isopropyl alcohol | 234.800 |
| Colloidal silicon dioxide | 8.800 |
| Magnesium stearate | 4.400 |
| Film-coating | |
| Ready mix film coating material | 35.200 |
| Total | 915.200 |

Procedure: Colesevelam hydrochloride was co-sifted with mannitol (Pearlitol SD 200) using 20# S. S. Sieve and transferred into Uniglatt fluid bed processor and mixed for 10 minutes. Wet granulation solution was prepared by dissolving povidone in a mixture of isopropyl alcohol and water. The wet granulation solution was sprayed on to the Colesevelam-mannitol mixture in the Uniglatt by top spray mechanism. The mass was dried at temperature of 50° C. to 60° C. for sufficient time till loss on drying value of about 8% to 12% was achieved. The dried granules were sifted through 100# Sieve. Over sized granules were milled using ball mill and the milled mass was sifted through 100# sieve. The sifted granules were blended with presifted colloidal silicon dioxide (sifted through 40#) and magnesium stearate (sifted through 60#) in a conta blender and compressed on 0.75×0.35 inch elliptical punches and dies to obtain tablet having weight of 880 mg per tablet and hardness of about 80 to 200 N. The compressed tablets were coated using coating solution prepared by dissolving coating ready mix containing HPMC 5 cps and Triacetin in water, till weight gain of 4.0% of the core tablets (weight of coated tablets 915.200 mg) was achieved.

### Example 3

| Ingredients | Quantity (g) |
|---|---|
| Core | |
| Colesevelam hydrochloride | 156.250 |
| Mannitol | 25.557 |
| Purified water | 13.980 |
| Ethyl cellulose | 13.813 |
| Isopropyl alcohol | 55.250 |
| Colloidal silicon dioxide | 2.125 |
| Magnesium stearate | 1.063 |
| Film-coating | |
| Ready mix film coating material | 8.800 |
| Total | 221.300 |

Procedure: Colesevelam hydrochloride was co-sifted with mannitol (Pearlitol SD 200) using 20# S. S. Sieve and transferred into a rapid mixer granulator and mixed for 5 minutes at 100 rpm. Water was added into the rapid mixer granulator and mixed for about 3 minutes at impeller speed of 100 rpm. Wet granulation solution was prepared by dissolving ethyl cellulose in isopropyl alcohol by warming at about 45°-50° C. The binder solution at 45°-50° C. was added to the Colesevelam-mannitol mixture and mixed at impeller high speed 180 to 200 rpm with chopper off condition for sufficient time till a cohesive mass is formed. The mass was air dried for sufficient time in Glatt drier and further dried at temperature of 50 to 60° C. till loss on drying value of about 7% to 10% was achieved. The dried granules were sifted through 100# Sieve. Over sized granules were milled using ball mill and the milled mass was sifted through 100# sieve. The sifted granules were blended with presifted colloidal silicon dioxide (sifted through 40#) and magnesium stearate (sifted through 60#) in a conta blender and compressed on 0.75×0.35 inch elliptical punches and dies to obtain tablet having weight of 880 mg per tablet and hardness of about 80 to 200 N. The compressed tablets were coated using coating solution prepared by dissolving coating ready mix containing HPMC 5 cps and Triacetin in water, till weight gain of 4.0% of the core tablets (weight of coated tablets 885.200 mg) was achieved.

### Example 4

| Ingredients | Quantity (g) |
|---|---|
| Core | |
| Colesevelam hydrochloride | 156.250 |
| Mannitol | 15.787 |
| Polyvinyl pyrrolidone (Povidone 30) | 7.500 |
| Purified water | 19.000 |
| Ethyl cellulose | 13.813 |
| Isopropyl alcohol | 58.000 |
| Colloidal silicon dioxide | 5.525 |
| Magnesium stearate | 2.125 |
| Film-coating | |
| Ready mix film coating material | 8.800 |
| Total | 228.810 |

Procedure: Colesevelam hydrochloride was co-sifted with mannitol (Pearlitol SD 200) using 20# S. S. Sieve and transferred into a rapid mixer granulator and mixed for 5 minutes at 100 rpm. The binder solution was prepared by dissolving povidone in water. The povidone solution was added into the rapid mixer granulator and was mixed for about 5 minutes at impeller speed 100 rpm. The second binder solution was prepared by dissolving ethyl cellulose in isopropyl alcohol by warming at about 45°-50° C. The binder solution at 45°-50° C. was added to the Colesevelam-mannitol mixture and mixed at impeller high speed 180 to 200 rpm with chopper off condition for sufficient time till a cohesive mass is formed. The mass was air dried for sufficient time in Glatt drier and further dried at temperature of 50 to 60° C. till loss on drying value of about 8% to 12% was achieved. The dried granules were sifted through 100# Sieve. Over sized granules were milled using ball mill and the milled mass was sifted through 100# sieve. The sifted granules were blended with presifted colloidal silicon dioxide (sifted through 40#) and magnesium stearate (sifted through 60#) in a conta blender and compressed on 0.75×0.35 inch elliptical punches and dies to obtain tablet having weight of 880.04 mg per tablet and hardness of about 80 to 200 N. The compressed tablets were coated using coating solution prepared by dissolving coating ready mix containing HPMC 5 cps and Triacetin in purified water, till weight gain of 4.0% of the core tablets (weight of coated tablets 915.240 mg) was achieved.

### Example 5

| Ingredients | Quantity (g) |
|---|---|
| Core | |
| Colesevelam hydrochloride | 156.250 |
| Mannitol | 13.950 |
| Polyethylene glycol 6000 | 5.000 |
| Purified water | 19.000 |
| Polyvinyl pyrrolidone (Povidone 30) | 22.500 |
| Isopropyl alcohol | 58.000 |
| Colloidal silicon dioxide | 2.200 |
| Magnesium stearate | 1.100 |
| Film-coating | |
| Ready mix film coating material | 8.800 |
| Total | 228.800 |

Procedure: Colesevelam hydrochloride was co-sifted with mannitol (Pearlitol SD 200) using 20# S. S. Sieve and transferred into a rapid mixer granulator and mixed for 5 minutes at 100 rpm. The wetting solution was prepared by dissolving polyethylene glycol 6000 in water. This wetting solution was added into the rapid mixer granulator and mixed for about 5 minutes at impeller speed of 100 rpm. The binder solution was prepared by dissolving povidone in a mixture of isopropyl alcohol and water. Wet granulation solution was added to the Colesevelam-mannitol mixture and mixed at impeller high speed 180 to 200 rpm with chopper off condition for sufficient time till a cohesive mass was formed. The mass was air dried for sufficient time in Glatt drier and further dried at temperature of 50 to 60° C. till loss on drying value of about 8% to 12% was achieved. The dried granules were sifted through 100# Sieve. Over sized granules were milled using ball mill and the milled mass was sifted through 100# sieve. The sifted granules were blended with presifted colloidal silicon dioxide (sifted through 40#) and magnesium stearate (sifted through 60#) for about 5 minutes in a conta blender and compressed on 0.75×0.35 inch elliptical punches and dies to obtain tablet having weight of 880 mg per tablet and hardness of about 80 to 200 N. Compressed tablets were coated using coating solution prepared by dissolving coating ready mix containing HPMC 5 cps and Triacetin in water, till weight gain of 4.0% of the core tablets (weight of coated tablets 915.200 mg) was achieved.

### Example 6

| Ingredients | Quantity (g) |
|---|---|
| Core | |
| Colesevelam hydrochloride | 625.000 |
| Mannitol (Pearlitol SD 200) | 90.000 |
| Croscarmellose Sodium (Ac-di-sol) | 50.000 |
| Ethyl cellulose (Ethocel N 7 Pharm) | 80.000 |
| Purified water | 105.000 |
| Isopropyl alcohol | 400.000 |
| Colloidal silicon dioxide | 6.260 |
| Magnesium stearate | 2.680 |
| Film-coating | |
| Ready mix film coating material | 60 |

Procedure: Colesevelam hydrochloride was co-sifted with mannitol (Pearlitol SD 200) using 20# stainless steel sieve, Croscarmellose sodium using 40# stainless steel sieve and transferred into a rapid mixer granulator (capacity 3 L) and mixed for 5 minutes at 100 rpm. Wet granulation solution was prepared by dissolving Ethyl cellulose (Ethocel N 7 Pharm) in isopropyl alcohol. Water was added to the Colesevelam-mannitol-Croscarmellose sodium mixture and mix for 5 minutes at impeller 100 rpm. Wet granulation solution was added to the Colesevelam-mannitol-Croscarmellose sodium-water mixture and mixed at impeller high speed 180 to 200 rpm with chopper off condition for sufficient time till a highly cohesive mass was formed. The mass was air dried for sufficient time in fluid bed drier and further dried at temperature of 50° C. to 60° C. till loss on drying of about 8% to 12% was achieved. Dried granules were sifted through 100# Sieve. Over sized granules were milled using ball mill and the milled mass was sifted through 100# Sieve. Sifted granules were blended with presifted colloidal silicon dioxide (sifted through 40#) and magnesium stearate (sifted through 60#) in a conta blender. The blend was compressed on 0.75×0.35 inch elliptical punches and dies to obtain tablet having weight of 959 mg per tablet and hardness of about 80 to 200 N. Compressed tablets were coated using coating solution prepared by dissolving coating ready mix containing HPMC 5 cps and Triacetin in water, till weight gain of 4.0 to 6.0% of the core tablets was achieved.

### Example 7

| Ingredients | Quantity (g) |
|---|---|
| Core | |
| Colesevelam hydrochloride | 1875.000 |
| Mannitol (Pearlitol SD 200) | 288.000 |
| Croscarmellose Sodium (Ac-di-sol) | 150.000 |
| Polyvinylpyrrolidone (Povidone K-30) | 222.000 |
| Purified water | 315.000 |
| Isopropyl alcohol | 1000 |
| Colloidal silicon dioxide | 21 |
| Magnesium stearate | 9.000 |
| Film-coating | |
| Ready mix film coating material | 172.800 |

Procedure: colesevelam hydrochloride was co-sifted with mannitol (Pearlitol SD 200) using 20# stainless steel sieve, Croscarmellose sodium using 40# stainless steel sieve and transferred into a rapid mixer granulator (capacity 10 L) and mixed for 5 minutes at 100 rpm. Wet granulation solution was prepared by dissolving Povidone K-30 in mixture of isopropyl alcohol and water. The binder solution was added to the Colesevelam-mannitol-Croscarmellose sodium mixture and mixed at impeller high speed of 180 to 200 rpm with chopper off condition for sufficient time till a highly cohesive mass was formed. The mass was air dried for sufficient time in Fluid bed drier and further dried at temperature of 50° C. to 60° C. till loss on drying of about 8% to 12% was achieved. Dried granules were sifted through 100# Sieve. Over sized granules were milled using ball mill and the milled mass was sifted through 100# Sieve. Sifted granules were blended with presifted colloidal silicon dioxide (sifted through 40#) and magnesium stearate (sifted through 60#) in a conta blender. The blend was compressed on 0.75×0.35 inch elliptical punches and dies to obtain tablet having weight of 960 mg per tablet and hardness of about 80 to 200 N. Compressed tablets were coated using coating solution prepared by dissolving coating ready mix containing HPMC 5 cps and Triacetin in water, till weight gain of 4.0% to 6.0% of the core tablets was achieved.

### Example 8

| Ingredients | Quantity (g) |
|---|---|
| Core | |
| Colesevelam hydrochloride | 625.000 |
| Mannitol (Pearlitol SD 200) | 96.000 |
| Croscarmellose Sodium (Ac-di-sol) | 50.000 |
| Polyvinylpyrrolidone (Povidone K-30) | 74.000 |
| Purified water | 105.000 |
| Isopropyl alcohol | 450.000 |
| Colloidal silicon dioxide | 7.000 |
| Magnesium stearate | 3.000 |
| Film-coating | |
| Ready mix film coating material | 57.600 |

Procedure: Colesevelam hydrochloride was co-sifted with mannitol (Pearlitol SD 200) using 20# stainless steel sieve, Croscarmellose sodium using 40# stainless steel sieve and transferred into Uniglatt (Fluid bed processor) (capacity 3 L) and fluidized for 5 minutes. Wet granulation solution was prepared by dissolving Povidone K-30 in mixture of isopropyl alcohol and water. The wet granulation solution was sprayed onto the Colesevelam-mannitol-Croscarmellose sodium mixture till a granular wet mass was formed. The mass was air dried for sufficient time and further dried at temperature of 50° C. to 60° C. till loss on drying value of about 8% to 12% was achieved. The dried granules were sifted through 100# Sieve. The over sized granules were milled using ball mill and the milled mass was sifted through 100# Sieve. The sifted granules were blended with presifted colloidal silicon dioxide (sifted through 40#) and magnesium stearate (sifted through 60#) in a conta blender. The blend was compressed on 0.75×0.35 inch elliptical punches and dies to obtain tablet having weight of 960 mg per tablet and hardness of about 80 to 200 N. The compressed tablets were coated using coating solution prepared by dissolving coating ready mix containing HPMC 5 cps and Triacetin in water, till weight gain of 4.0 to 6.0% of the core tablets was achieved.

### Example 9

### Preparation of Sevelamer Hydrochloride

50 g Poly(allylamine hydrochloride) and 75 ml water were mixed at 25 to 35° C. to get a clear solution. The solution was further cooled to 5 to 15° C. and 13.68 g sodium hydroxide solution in water (65-70% of sodium hydroxide pellets by mole of Polyallylamine hydrochloride) was added to the reaction mass at 5 to 15° C. and stirred for 30 minutes, 400 ml toluene and 2 ml SPAN-85 were added to it at 5 to 15° C. The temperature of the reaction mixture was then raised to 20 to 25° C. and maintained for 15 min. The reaction mixture was filtered to remove any extraneous matter at 25 to 35° C. The temperature of the filtrate was further raised to 55 to 60° C. and maintained for 15 minutes. 4.5 g epichlorohydrin was added at constant temperature of 55 to 60° C. to reaction mixture and maintained for 3 hr at 55 to 60° C. The reaction mixture was cooled to 25 to 35° C. and product was isolated by centrifugation. The wet cake was further sludged with water (3×750 ml) for 45 min at 25 to 50° C., filtered and dried in FBD at 25 to 90° C.

Chloride content—4.45 meq/gm
Phosphate binding capacity by IC method—5.97 mmol/gm.
Degree of cross linking—16.4%

### Yield—77.0% w/w

### Example 10

### Preparation of 6-bromohexyltrimethylammonium bromide

1 kg 1,6-dibromohexane was stirred in 3 L of tetrahydrofuran (THF) and 242 g of trimethylamine was slowly purged into reaction mass at −10 to 0° C. for 2-3 hrs. The reaction mass was further stirred for 48 hrs and the separated solid was immediately filtered, washed with 2 L tetrahydrofuran, suck dried and then dried in vacuum oven at 40-45° C. till constant weight was obtained. 1.047 kg 6-bromohexyltrimethylammonium bromide was obtained.

### Yield—104% w/w

### Example 11

### Preparation of 6-Bromohexyltrimethylammonium Bromide

1 kg 1,6-dibromohexane was stirred in 3 L tetrahydrofuran (THF) and 242 gm trimethylamine was slowly purged into reaction mass at −10 to 0° C. for 2-3 hrs. The reaction mass was stirred for 3 hrs and the separated solid was immediately filtered and washed with 2 L tetrahydrofuran (THF). The material thus obtained was suck dried first and then dried in vacuum oven at 40-45° C. till constant weight was obtained. 783 g 6-bromohexyltrimethylammonium bromide was obtained.

### Yield—78.3% w/w

### Example 12

30 g of Sevelamer hydrochloride, 800 ml methanol, 31.6 g 6-bromohexyltrimethylammonium bromide and 18.9 g 1-bromodecane were heated at 65° C. 18 gm 50% (w/w) aqueous sodium hydroxide solution was added and continued heating for 18 hrs. The reaction mass was cooled to room temperature and filtered. The wet cake thus obtained was stirred in 2.0 L methanol for 30 minutes and filtered. Methanol washing was repeated for one more time. Wet cake then stirred in 1.1 L (2 M) sodium chloride solution in water for 30 minutes and filtered. The material was further stirred in 2.0 L (2 M) sodium chloride solution in water for 30 minutes and filtered. The wet cake thus obtained was stirred in 0.8 L purified water for 30 minutes and filtered. Water washings were repeated for two more times. The wet cake was then dried and analysed.

Chloride content: 9.0%

### Example 13

30 g of Sevelamer hydrochloride, 800 ml methanol, 31.6 g 6-bromohexyltrimethylammonium bromide and 18.9 g 1-bromodecane were heated at 65° C. 4.5 gm 50% (w/w) aqueous sodium hydroxide solution was added and continued heating for 2 hrs. Successive three additions of 4.5 gm 50% (w/w) aq. sodium hydroxide was added each after every 2 hrs. Reaction was then continued for 12 hrs at same temperature. The reaction mass was cooled to room temperature and filtered. The wet cake thus obtained was stirred in 2.0 L methanol for 30 minutes and filtered. Methanol washing was repeated for one more time. Wet cake then stirred in 1.1 L (2 M) sodium chloride solution in water for 30 minutes and filtered. The material was further stirred in 2.0 L (2 M) sodium chloride solution in water for 30 minutes and filtered. The wet cake thus obtained was stirred in 0.8 L purified water for 30 minutes and filtered. Water washings were repeated for two more times. The wet cake was then dried and analysed.

### Example 14

50 g of Sevelamer hydrochloride, 88 g 6-bromohexyltrimethylammonium bromide and 52.5 g 1-bromodecane were added to a solution of 22.5 gm of sodium hydroxide dissolved in 1.35 L methanol under stirring. The reaction mass was refluxed for 18 hrs. After the completion of reaction, the reaction mass was cooled to room temperature and filtered. Wet cake was stirred in 2.0 L methanol for 30 minutes and filtered. Again methanol washing was repeated. The wet cake thus obtained was stirred in 3.65 L (2 M) sodium chloride solution in water for 2 hrs and filtered. The material was further stirred in 3.65 L (2 M) sodium chloride solution in water for 2 hrs and filtered. The wet cake thus obtained was stirred in 3.65 L (0.003 N) HCl solution in water for 1 hr and filtered. The wet cake was further stirred twice in 3.65 L purified water for 1 hr and filtered, suck dried and further dried at 90-95° C. under vacuum either on rotary evaporator or fluidized bed dryer till constant weight of the material was obtained.

Chloride content: 14.33% (expected 18 to 26%)

### Example 15

50 g of Sevelamer hydrochloride, 88 g 6-bromohexyltrimethylammonium bromide and 52.5 g 1-bromodecane were added to a solution of 22.5 gm of sodium hydroxide dissolved in 1.35 L methanol under stirring. The reaction mass was refluxed for 18 hrs. After the completion of reaction, the reaction mass was cooled to room temperature and filtered. Wet cake was stirred in 2.0 L methanol for 30 minutes and filtered. Again methanol washing was repeated. The wet cake thus obtained was stirred in 3.65 L (2 M) sodium chloride solution in water for 2 hrs and filtered. The material was further stirred in 3.65 L (2 M) sodium chloride solution in water for 2 hrs and filtered. The wet cake thus obtained was stirred in 3.65 L (0.003 N) HCl solution in water for 1 hr and filtered. Again the wet cake thus obtained was stirred in 1.8 L (0.003 N) HCl solution in water for 1 hr and filtered. The wet cake was further stirred twice in 3.65 L purified water for 1 hr and filtered, suck dried and further dried at 90-95° C. under vacuum either on rotary evaporator or fluidized bed dryer till constant weight of the material was obtained.

Chloride content: 17.58% (expected 18 to 26%)

### Example 16

### Preparation of Colesevelam Hydrochloride

50 g of Sevelamer hydrochloride, 88 g 6-bromohexyltrimethylammonium bromide and 52.5 g 1-bromodecane were added to a solution of 22.5 gm of sodium hydroxide dissolved in 1.35 L methanol under stirring. The reaction mass was refluxed for 22 hrs. After the completion of reaction, the reaction mass was cooled to room temperature and filtered. The wet cake thus obtained was stirred in 3.65 L (2 M) sodium chloride solution in water for 2 hrs and filtered. The material was further stirred in 3.65 L (2 M) sodium chloride solution in water for 2 hrs and filtered. The wet cake thus obtained was stirred in 3.65 L (0.1 N) HCl solution in water for 1 hr and filtered. The wet cake was further stirred twice in 3.65 L purified water for 1 hr (multiple washings may require to get rid of inorganic matrix depending upon the batch size and agitation of the product), filtered, suck dried and further dried at 90-95° C. under vacuum either on rotary evaporator or fluidized bed dryer till constant weight of the material was obtained. 99 gm Colesevelam hydrochloride was obtained.

Bile binding capacity—4.12 mmol/g. Yield—198% w/w

### Example 17

### Preparation of Colesevelam Hydrochloride

50 g of Sevelamer hydrochloride, 88 g 6-bromohexyltrimethylammonium bromide and 52.5 g 1-bromodecane were added to a solution of 22.5 g sodium hydroxide dissolved in 1.35 L methanol and the reaction mass was refluxed for 6 hrs. Then reaction mass was cooled to room temperature and filtered the material. The wet cake obtained was stirred in 3.65 L (2M) sodium chloride solution in water for 2 hrs twice and filtered. The wet cake thus obtained was further stirred in 3.65 L (0.1 N) HCl solution in water for 1 hr and filtered. The material was stirred in 3.65 L purified water for 1 hr twice, filtered, suck dried and then dried at 90-95° C. under vacuum either on rotary evaporator or fluidized bed dryer till constant weight of the material was obtained. 97 gm Colesevelam hydrochloride was obtained.

Bile binding capacity—3.93 mmol/g and Yield—194% w/w

### Example 18

### Preparation of Colesevelam Hydrochloride

To a solution of 22.5 gm sodium hydroxide dissolved in 1.35 L methanol, 50 gm of Sevelamer hydrochloride, 88 gm 6-bromohexyltrimethylammonium bromide and 52.5 gm 1-bromodecane were added and the reaction mass was refluxed for 12 hrs. Then the reaction mass was cooled to room temperature and filtered. The wet cake obtained was stirred in 3.65 L (2M) sodium chloride solution in water for 2 hrs twice and filtered. The obtained wet cake was stirred in 3.65 L (0.1 N) HCl solution in water for 1 hr, filtered and stirred in 3.65 L purified water for 1 hr twice and was filtered. The material thus obtained was suck dried and then dried at 90-95° C. under vacuum either on rotary evaporator or fluidized bed dryer till constant weight of the material was obtained. 102 g Colesevelam hydrochloride was obtained. Bile binding capacity-3.66 mmol/g and Yield-204% w/w.

### Example 19

### Preparation of Colesevelam Hydrochloride

50 g of Sevelamer hydrochloride, 88 g 6-bromohexyltrimethylammonium bromide and 52.5 g 1-bromodecane were added to a solution of 22.5 g sodium hydroxide dissolved in 1.35 L methanol and the reaction mass was refluxed for 4 hrs. After the completion of reaction the reaction mass was cooled to room temperature and filtered. The obtained wet cake was stirred twice in 3.65 L (2 M) sodium chloride solution in water for 2 hrs and filtered. The wet cake was further stirred in 3.65 L (0.1 N) HCl solution in water for 1 hr and the obtained material was filtered. The wet cake was further stirred in 3.65 L purified water for 1 hr and filtered and again stirred in 3.65 L purified water for 1 hr. The material was filtered, suck dried and further dried at 90-95° C. under vacuum either on rotary evaporator or fluidized bed dryer till constant weight of the material was obtained. 90 g Colesevelam hydrochloride was obtained.

Bile binding capacity—4.65 mmol/g and Yield—180% w/w

### Example 20

### Preparation of Colesevelam Hydrochloride

50 g of Sevelamer hydrochloride, 88 g 6-bromohexyltrimethylammonium bromide and 52.5 g 1-bromodecane were added to a solution of 22.5 g sodium hydroxide dissolved in 1.35 L methanol and the reaction mass was refluxed for 29 hrs. Then reaction mass was cooled to room temperature and filtered. The wet cake obtained was stirred in 3.65 L (2M) sodium chloride solution in water for 2 hrs and filtered. The obtained material was again stirred in 3.65 L (2M) sodium chloride solution in water for 2 hrs and filtered. The wet cake thus obtained was stirred in 3.65 L (0.1 N) HCl solution in water for 1 hr, filtered and further stirred twice in 3.65 L purified water for 1 hr and filtered. The material thus obtained was first suck dried and then dried at 90-95° C. under vacuum either on rotary evaporator or fluidized bed dryer till constant weight of the material was obtained. 92 g Colesevelam hydrochloride was obtained.

Bile binding capacity—3.64 mmol/g and Yield—184% w/w

### Example 21

### Preparation of Colesevelam Hydrochloride

To a solution of 11.25 gm sodium hydroxide dissolved in 0.675 L methanol, 25 gm Sevelamer hydrochloride, 44 gm 6-bromohexyltrimethylammonium bromide were added and the reaction mass was refluxed for 1 hr. 26.25 gm 1-bromodecane was added to the reaction mass at reflux temperature and stirred at reflux for 18 hrs. Then the reaction mass was cooled to room temperature and filtered. The wet cake was stirred in 1.82 L (2M) sodium chloride solution in water for 2 hrs and the material was filtered off and this is repeated again and the material was filtered. The wet cake obtained was stirred in 1.82 L (0.1 N) HCl solution in water for 1 hr and filtered off. The material obtained was stirred in 1.82 L purified water for 1 hr and the material was filtered. This step was repeated again. The material obtained was first suck dried and then dried at 90-95° C. under vacuum either on rotary evaporator or fluidized bed dryer till constant weight of the material was obtained. 52.5 gm Colesevelam hydrochloride was obtained.

Bile binding capacity 3.43 mmol/gm and Yield—210% w/w

### Example 22

### Preparation of Colesevelam Hydrochloride

11.25 g sodium hydroxide was dissolved in 0.675 L methanol under stirring and 25 g Sevelamer hydrochloride, 44 g 6-bromohexyltrimethylammonium bromide were added and the reaction mass was refluxed for 30 min. 26.25 g 1-bromodecane was added to the obtained reaction mass at reflux temperature and stirred at reflux for 18 hrs. The reaction mass was cooled to room temperature and filtered. The wet cake obtained was stirred in 1.82 L (2M) sodium chloride solution in water for 2 hrs and filtered. The sodium chloride treatment was repeated once more and the obtained material was stirred in 1.82 L (0.1 N) HCl solution in water for 1 hr. Material was filtered and stirred in 1.82 L purified water for 1 hr, twice. The material was filtered, suck dried and then dried at 90-95° C. under vacuum either on rotary evaporator or fluidized bed dryer till constant weight of the material is obtained. 52.5 g Colesevelam hydrochloride was obtained.

Bile binding capacity—3.58 mmol/g and Yield—210% w/w

### Example 23

### Preparation of Colesevelam Hydrochloride

11.25 gm sodium hydroxide was dissolved in 0.675 L methanol under stirring and 25 gm Sevelamer hydrochloride, 44 gm 6-bromohexyltrimethylammonium bromide were added and the reaction mass was refluxed for 2 hrs. 26.25 g 1-bromodecane was added to the reaction mixture at reflux temperature and stirred at reflux for 18 hr. The reaction mass was cooled to room temperature and filtered. The wet cake was stirred in 1.82 L (2M) sodium chloride solution in water for 2 hrs twice and the material was filtered. The wet cake obtained was stirred in 1.82 L (0.1 N) HCl solution in water for 1 hr and filtered. The obtained material was again stirred in 1.82 L purified water for 1 hr twice. The material obtained was filtered, suck dried and further dried at 90-95° C. under vacuum either on rotary evaporator or fluidized bed dryer till constant weight of the material was obtained. 53.5 g Colesevelam hydrochloride was obtained.

Bile binding capacity 3.55 mmol/g and Yield—214% w/w

### Example 24

### Preparation of Colesevelam Hydrochloride

22.5 g sodium hydroxide was dissolved in 1.35 L methanol under stirring and 50 g Sevelamer hydrochloride, 88 g 6-bromohexyltrimethylammonium bromide and 64.14 gm 1-bromodecane were added and refluxed for 22 hrs. The reaction mass was cooled to room temperature and filtered. The wet cake was stirred in 3.65 L (2M) sodium chloride solution in water for 2 hrs twice and filtered. The material was further stirred in 3.65 L (0.1 N) HCl solution in water for 1 hr and filtered. The obtained material was stirred in 3.65 L purified water for 1 hr twice and filtered. The material obtained was suck dried and then dried at 90-95° C. under vacuum either on rotary evaporator or fluidized bed dryer till constant weight of the material was obtained. 90 g Colesevelam hydrochloride was obtained. Bile binding capacity—3.32 mmol/g and Yield—180% w/w

### Example 25

### Preparation of Colesevelam Hydrochloride

31 gm sodium hydroxide was dissolved in 1.35 L methanol under stirring and 88 gm 6-bromohexyltrimethylammonium bromide and 64.14 gm 1-bromodecane were added. This reaction mass was refluxed for 22 hrs. Then reaction mass was cooled to room temperature and filtered. The wet cake was stirred in 3.65 L (2 M) sodium chloride solution in water for 2 hrs and filtered. 3.65 L (2 M) sodium chloride solution in water was added to the obtained material for 2 hr and filtered. The wet cake was stirred in 3.65 L (0.1 N) HCl solution in water for 1 hr and filtered. The obtained material was stirred in 3.65 L purified water twice for 1 hr and filtered. The material was suck dried and dried at 90-95° C. under vacuum either on rotary evaporator or fluidized bed dryer till constant weight of the material was obtained. 90 gm Colesevelam hydrochloride was obtained.

Bile binding capacity—3.64 mmole/gm and Yield—180% w/w

While the present invention is described above in connection with preferred or illustrative embodiments, these embodiments are not intended to be exhaustive or limiting of the invention. Rather, the invention is intended to cover all alternatives, modifications and equivalents included within its spirit and scope, as defined by the appended claims.

1-43. (canceled)

44. A pharmaceutical composition comprising wet granulated bile acid sequestrant and at least one pharmaceutical excipient, wherein the composition is free of reducing sugar.

45. The composition as claimed in claim 44, wherein the bile acid sequestrant is selected from the group consisting of Colesevelam, Cholestyramine, Colestipol, Sevelamer and Colestimide.

46. The composition as claimed in claim 44, wherein the bile acid sequestrant is an alkylated cross-linked polymer.

47. A pharmaceutical composition comprising wet granulated Colesevelam along with at least one pharmaceutically acceptable excipient, wherein the particles of Colesevelam are spherical or globular in shape.

48. A pharmaceutical composition comprising wet granulated Colesevelam or pharmaceutically acceptable salts thereof and at least one pharmaceutical excipient.

49. The composition as claimed in claim 47 or claim 48 wherein Colesevelam is present from about 50% to about 85% by weight of the total composition.

50. The composition as claimed in claim 47 or claim 48, wherein the composition comprises not more than 90% by weight of hydrated Colesevelam.

51. The composition as claimed in claim 47 or claim 48, wherein the unit dose strength is from about 500 mg to about 800 mg of Colesevelam.

52. The composition as claimed in claim 49 or claim 50, wherein the composition is free of reducing sugars.

53. The composition as claimed in claim 47 or claim 48, wherein said Colesevelam has one or more characteristics selected from group consisting of: bile binding capacity (BBC) not less than 0.5 mM/gm when measured by method selected from Sodium Glycochenodeoxycholate hydrate method or Sodium Glycocholate hydrate method or Sodium Taurodeoxycholate hydrate method; chloride content in the range of 3.0 to 8.0 meq/gm; bromide content not more than 0.5% w/w and at least 50% particles having particle size less than 1000 microns.

54. The composition as claimed in claim 45 or claim 52, wherein the composition is in the form of granules, tablets or capsules.

55. The composition as claimed in claim 44 further comprising an additional agent selected from biguanide, sulfonyl urea, thiazolidinedione, dipeptidyl peptidase IV inhibitor, alpha-glucosidase inhibitor, meglitinides, fibrates and statins.

56. A pharmaceutical composition comprising wet granulated Colesevelam in combination with metformin or pharmaceutically acceptable salts thereof.

57. The composition as claimed in claim 56, wherein metformin or its pharmaceutically acceptable salt is present in an amount from 250 mg to 2000 mg and Colesevelam or its pharmaceutically acceptable salt is present in an amount from 500 mg to 4000 mg.

58. The composition as claimed in claim 52 or claim 55, wherein the composition is safe for administration to a diabetic patient.

59. A pharmaceutical composition comprising wet granulated Colesevelam or pharmaceutically acceptable salts thereof and at least one pharmaceutical excipient; wherein the composition comprises at least one polyol selected from the group consisting of inositol, sorbitol, mannitol, isomalt, xylitol, lactitol, erythritol and maltitol.

60. A process for preparation of wet granulated bile acid sequestrant composition as defined in claim 1, comprising the steps of:
(a) providing a bile acid sequestrant;
(b) providing a wet-granulation solution said wet-granulation solution comprising at least about 70% (w/w) of organic solvent, said wet-granulation solution having not more than about 25% (w/w) of water;
(c) wet granulating the bile acid sequestrant with the wet-granulation solution to form granules and optionally formulating the resulting granules into final dosage form.

61. A process for preparation of wet granulated Colesevelam comprising the steps of:
(a) providing the Colesevelam;
(b) providing a wet-granulation solution said wet-granulation solution comprising at least about 70% (w/w) of organic solvent, said wet-granulation solution having not more than about 25% (w/w) of water;
(c) wet granulating the Colesevelam with the wet-granulation solution to form granules and optionally formulating the resulting granules into final dosage form.

62. The process as claimed in claim 60 or claim 61, wherein the wet granulation comprises high shear granulation or spray granulation.

63. The process as claimed in claim 61, further comprising mixing said Colesevelam with at least one diluent before granulation.

64. The process as claimed in claim 61, wherein said Colesevelam is pre-wetted using water or an aqueous solution of polyethylene glycol before said granulation.

65. The process as claimed in claim 60 or claim 61, wherein said organic solvent is a lower-chain alcohol selected from the group consisting of ethyl alcohol and isopropyl alcohol.

66. The process as claimed in claim 62, wherein said process comprises the use of at least one binder selected from the group consisting of: hydroxy propyl methylcellulose, hydroxy propyl cellulose, hydroxy ethyl cellulose, ethyl cellulose, cellulose derivatives, polyvinylpyrrolidone, polyethylene glycol and maize starch.

67. The process as claimed in claim 66, wherein the binder is ethyl cellulose or polyvinylpyrrolidone.

68. The process as claimed in claim 61, wherein said wet granulation comprises the steps of:
(a) preparing a mixture of Colesevelam or pharmaceutically acceptable salts thereof and one or more diluents and optionally disintegrants;
(b) optionally wetting the mixture of step (a) using water or solution of polyethylene glycol;
(c) preparing a wet granulation solution by dissolving the binder in a mixture of organic solvent and water;
(d) granulating the mixture of step (a) or step (b) using wet granulation solution by high shear granulation or spray granulation to form granulated mass;
(e) drying the granulated mass;
(f) milling the dried granulated mass using ball mill or fluid energy mill to form granules of suitable size;
(g) lubricating the milled granules;
(h) compressing the lubricated granules into tablets or filling the lubricated granules into capsules;
(i) optionally coating the compressed tablets.

69. The process as claimed in claim 68, wherein the Colesevelam is Colesevelam hydrochloride having chloride content in the range of 3.0 to 8.0 meq/gm and bromide content less than 0.5% w/w.

70. A process for preparation of Colesevelam hydrochloride having chloride content in the range of 3.0 to 8.0 meq/gm and bromide content less than 0.5% w/w comprising treating alkylated crosslinked polymer with hydrochloric acid to get the product.

71. A process for preparation of crosslinked polymer or salt thereof comprising treating crosslinked polymer or salts thereof with dilute hydrochloric acid to get the product having chloride content in the range of 3-8 meq/gm preferably 4-8 meq/gm.

72. The process as claimed in claim 71, wherein the dilute hydrochloric acid has a concentration less than about 5N.

73. The process as claimed in claim 71, wherein said crosslinked polymer is selected from Sevelamer, Colesevelam or salts thereof.

74. The process as claimed in claim 70, wherein the alkylated crosslinked polymer is prepared by a process comprising: a) alkylating cross linked polyallylamine polymer or salt thereof with 6-bromohexyltrimethylammonium bromide and 1-bromodecane in presence of alcoholic solvent.

75. The process as claimed in claim 74, wherein the 6-bromohexyltrimethylammonium bromide is prepared by treating 1,6-dibromohexane with trimethyl amine at low temperature in the mole ratio of 1:0.9 to 1:1.2.

76. The process as claimed in claim 74, wherein said alkylation is carried out in the presence of a base selected from the group consisting of sodium hydroxide, potassium hydroxide, lithium hydroxide or mixture thereof and wherein said alcoholic solvent is selected from the group consisting of methanol, ethanol, n-propanol, isopropanol, n-butanol or mixture thereof.

77. The process as claimed in claim 74, wherein said alkylation is carried out at temperature range of 25 to 90° C.

78. The process as claimed in claim 74, wherein the alkylated crosslinked polymer obtained is further washed with sodium chloride solution or purified water to remove the inorganic matrix.

79. The process as claimed in claim 78, wherein said sodium chloride solution is in the range of 0.1 to 10 M in water.

80. The process as claimed in claim 70, wherein said product is dried in air tray dryer (ATD) or vacuum tray dryer (VTD) or fluidized bed dryer (FBD) or rotary evaporator for 1 to 48 hours at temperature of about 50-110° C. to get loss on drying (LOD) less than 10%.

81. Colesevelam hydrochloride having one or more characteristics selected from group consisting of: bile binding capacity (BBC) not less than 0.5 mM/gm; chloride content in the range of 3.0 to 8.0 meq/gm; bromide content not more than 0.5% w/w and at least 50% particles having particle size less than 1000 microns.

82. Colesevelam hydrochloride with bromide content not more than 0.5% w/w.

83. Colesevelam hydrochloride as claimed in claim 81, wherein said bile binding capacity (BBC) is not less than 0.5 mM/gm when measured by method selected from Sodium Glycochenodeoxycholate hydrate method or Sodium Glycocholate hydrate method or Sodium Taurodeoxycholate hydrate method.

84. A pharmaceutical composition comprising wet granulated Colesevelam hydrochloride and at least one pharmaceutical excipient, wherein said Colesevelam hydrochloride is prepared by the process as claimed in claim 74.

85. A method for removing the bile acids from a patient in need thereof comprising the step of administering to the patient a composition as claimed in claim 44 containing a therapeutically effective amount of bile acid sequestrant.
